(19) United States
(12) Patent Application Publication
(10) Pub. No.: US 2019/0038529 A1
(43) Pub. Date: Feb. 7, 2019

(54) ODOR-REDUCING DEVELOPER FOR OXIDATION DYES II

(71) Applicant: Henkel AG & Co. KGaA, Duesseldorf (DE)

(72) Inventors: Hartmut Manneck, Barnitz (DE); Stefan Hoepfner, Hamburg (DE); Matthias Schweinsberg, Langenfeld (DE); Astrid Kleen-Fehres, Hamburg (DE)

(73) Assignee: Henkel AG & Co. KGaA, Duesseldorf (DE)

(21) Appl. No.: 15/538,705

(22) PCT Filed: Nov. 2, 2015
(86) PCT No.: PCT/EP2015/075431

§ 371 Date: Jun. 22, 2017

(30) Foreign Application Priority Data
Dec. 22, 2014 (DE) 10 2014 226 750.1

(51) Int. Cl. A61K 8/22; A61K 8/34; A61K 8/37; A61K 8/39; A61K 8/92; A61K 8/86; A61Q 5/10; A61Q 5/08

(52) U.S. Cl. CPC A61K 8/22; A61K 8/342; A61K 8/375; A61K 8/39; A61K 2800/882; A61K 8/92; A61K 8/86; A61Q 5/10; A61Q 5/08; A61K 8/345

(57) ABSTRACT

The aim of the present disclosure was to provide an improved oxidizing agent preparation for oxidative color modifying agents, which are present as soap based gels and contain ammonia as the alkalizing agent and optionally at least one oxidation dye precursor, and have a pH in the range of from about 8 to about 11.5, measured at 20° C. with which homogeneous and viscosity-stable application mixtures can be produced that release less ammonia during the entire leave-in time. It was surprisingly found that aqueous hydrogen peroxide preparations which contain selected 1-alkanols, ethylene glycol fatty acid esters, ethoxylated fatty alcohols, polyols and oils achieve said aim in a very satisfactory manner.

### CROSS-REFERENCE TO RELATD APPLICATION

This application is a U.S. National-Stage entry under 35 U.S.C. § 371 based on International Application No. PCT/EP2015/075431, filed Nov. 2, 2015 which was published under PCT Article 21(2) and which claims priority to German Application No. 102014226750.1, filed Dec. 22, 2014, which are all hereby incorporated in their entirety by reference.

### TECHNICAL FIELD

The present patent application relates to compositions that are suitable as oxidizing agents for soap gel-based hair lightener preparations and/or hair dye preparations, wherein the soap gel-based hair lightener preparations and/or hair dye preparations contain ammonia and/or ammonium hydroxide as the alkalizing agent and optionally at least one oxidative dye precursor and have a pH in the range of from about 8 to about 11.5, measured at 20° C.

### BACKGROUND

The application properties of the application mixture, in particular the release of ammonia and the odor of ammonia, can be reduced significantly with the oxidizing agents as contemplated herein.

Another subject matter of the present application relates to agents for changing the color of keratinic fibers, which can be prepared from two separate compositions by mixing the two compositions, wherein one of the two compositions is an oxidative composition according to the first subject matter of the application, and the second composition is a soap gel-based hair lightener and/or hair coloring preparation containing ammonia as an alkalizing agent and optionally at least one oxidative dye precursor and having a pH in the range of from about 8 to about 11.5, measured at 20° C., and also preferably containing, each based on its weight, from about 25-85% by weight water, also from about 5-20% by weight, preferably from about 8-16% by weight, of at least one salt of a C₁₂-C₂₂ fatty acid, preferably a salt of oleic acid, optionally from about 0.1-3% by weight of an anionic surfactant, selected from alkyl sulfates, alkyl ether sulfates and ether carboxylic acids with from about 10 to about 20 carbon atoms in the alkyl group and up to 16 glycol ether groups in the molecule, from about 2-20% by weight, preferably from about 3-16% by weight of at least one polyethylene glycol ether of a linear, saturated or unsaturated C₁₀-C₁₈ alkanol with 1 to 5 ethanol oxide units in the molecule, a total of from about 0-1% by weight of at least one oil, preferably at least one organic solvent, selected from monovalent to tetravalent C₂-C₆ alcohols, especially preferably in a total amount of from about 0.1-35% by weight and optionally at least one oxidation dye precursor, wherein all the quantitative amounts are based on the weight of the composition (A).

Another subject matter of the present disclosure relates to a kit for an oxidative color change of keratinic fibers comprising a soap gel-based, optionally dye-containing and ammonia-containing alkalizing preparation and an aqueous hydrogen peroxide preparation, wherein the hydrogen peroxide preparation is optimized to the extent that the ready-to-use mixture of alkalizing gel and hydrogen peroxide preparation constitutes a viscous cream or paste having a viscosity in the range of from about 2000 to about 5000 mPas (for example, measured at 20° C. with a model MV2 Haake viscometer at a rate of 8 revolutions per minute), which can be applied well to the fibers that are to be dyed or lightened and has a reduced release of ammonia during the application time of from about 5 to about 60 minutes.

Another subject matter of the present disclosure relates to a method for oxidative color change of keratinic fibers, wherein the ready-to-use lightener and/or coloring agent is prepared by mixing the components of the aforementioned kit immediately before use and then applying the preparation to the fibers, in particular hair, and rinsing it out again after a treatment time of from about 5 to about 60 minutes.

The present disclosure relates to an oxidative color change on keratinic fibers, in particular hair. Melanin, the endogenous coloring substance in hair, is destroyed to a certain extent in the treatment of keratinic fibers, in particular hair, using oxidizing agents, in particular hydrogen peroxide, so the fibers/hair are necessarily lightened, i.e., they change the color even in the absence of a coloring agent. Therefore, the term “color change” in the sense of the present patent application is understood to refer to lightening as well as dyeing using one or more coloring agents.

Those skilled in the art are familiar with various methods for changing the color of human hair. In general, either direct dyes or oxidative dyes are used for coloring human hair. Oxidative dyes are formed by oxidative coupling of one or more developer components to one another or to one or more coupler components. Coupler components and developer components are also known as oxidative dye precursors. Colorings produced using oxidative dyes are usually referred to as permanent or semipermanent colorings.

These agents usually contain hydrogen peroxide as the oxidizing agent. Since hydrogen peroxide in the alkaline pH range has inadequate stability in storage, oxidative dyes usually includes two components that are mixed together immediately prior to use. One component contains hydrogen peroxide in aqueous solution or emulsion, and this composition for stabilization of hydrogen peroxide has an acidic pH in the range of from about 2.5 to about 5.5. The second component contains one or more alkalizing agents in an amount such that the application mixture including the two components has a pH in the range of from about 8 to about 11. If the alkalizing preparation does not contain a dye or contains only small amounts of dye, the latter serves to mask unwanted hues, which may be formed in the oxidation of melanin, then this is a lightener or bleach. However, the alkalizing preparation may also contain oxidative dye precursors and/or direct dyes. The resulting application mixture then serves as a coloring agent. In addition, there are also coloring kits and coloring methods, in which the application mixture includes two components has a pH in the range of approx. about 6 to about 7.9. However, the coloring results of these so-called “acidic” colorings often do not achieve the quality that is obtained with alkaline application mixtures.

For oxidative coloring of hair, preferably alkalizing preparations containing ammonia are used, in particular in the North American market. These preparations are available as a soap-based gel and in particular contain large amounts of soap, i.e., salts of fatty acids and optionally other surfactants but little or no fatty alcohols for example. When applied to hair, such formulations have a very strong ammonia odor in comparison with alkaline bleach creams or coloring creams that contain fatty alcohol.

In addition, the alkalizing preparations contain ammonia as an alkalizing agent and optionally contain at least one oxidative dye precursor and have a pH in the range of from about 8 to about 11.5, measured at 20° C.

In addition, alkalizing preparations preferably containing, based on their weight, from about 25-85% by weight water, also from about 5-20% by weight, preferably from about 8-16% by weight of at least one salt of a C₁₂-C₂₂ fatty acid, preferably a salt of oleic acid, optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule, from about 2-20% by weight, preferably from about 3-16% by weight of at least one polyethylene glycol ether of a linear saturated or unsaturated C₁₀-C₁₈ alkanol with from about 1 to about 5 ethylene oxide units in the molecule, a total of from about 0-1% by weight of at least one oil, preferably at least one organic solvent selected from monovalent to tetravalent C₂-C₆ alcohols, especially preferably in a total amount of from about 0.1-35% by weight and optionally at least one oxidative dye precursor, wherein all the quantitative amounts are based on the weight of the composition (A).

For an oxidative hair color change, the alkalizing preparation is usually mixed with an aqueous oxidizing agent preparation, for example in a reclosable bottle or a shaking cup and the resulting application mixture is applied to the hair to be treated, where it remains for a treatment time of from about 5 to about 60 minutes before being rinsed off.

In the prior art, there are already various proposed solutions for reducing the ammonia odor of a hair treatment agent. For example, an alkanolamine, in particular a monoethanolamine, which is hardly volatile and does not have such a strong ammonia odor may be used instead of the ammonia. However, it has been found that the dye uptake capacity of the keratin fibers is best when ammonia is used. If the same color result is to be obtained with a monoethanolamine, then more hydrogen peroxide must be used or the application mixture must be left on the keratin fibers for a longer period of time. Ultimately the use of monoethanolamine also results in greater damage to the keratin fibers than ammonia.

Another possibility for reducing the odor of the application mixture is to mask the odor of ammonia with large amounts of perfume. However, this is difficult to achieve because of the very pungent odor of ammonia.

### BRIEF SUMMARY

Oxidation compositions for oxidative treatment of hair, kits for oxidative color change of keratinic fibers, and method for oxidative color change of keratinic fibers are provided herein. In an embodiment, an oxidation composition for oxidative treatment of hair includes from about 50-96% by weight water, from about 0.5-20% by weight hydrogen peroxide, at least one linear saturated 1-alkanol with 12-30 carbon atoms, at least one mono- or diester of ethylene glycol with palmitic acid and/or stearic acid as well as mixtures thereof, at least one ethoxylated fatty alcohol of the formula (III)

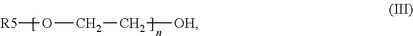

where R5 stands for a saturated or unsaturated, branched or unbranched C₈-C₂₄ alkyl group and n stands for an integer from 10 to 30, at least one ethoxylated fatty alcohol of formula (IV)

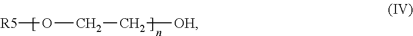

where R5 stands for a saturated or unsaturated, branched or unbranched C₈-C₂₄ alkyl group and n stands for an integer from about 50 to about 150, at least one polyol selected from C₂-C₉ alkanols with 2-6 hydroxyl groups, polyethylene glycols with 3-20 ethylene oxide units, or mixtures thereof, and at least one oil, wherein all the quantitative amounts are based on the weight of the oxidative composition.

In another embodiment, a kit for oxidative color change of keratinic fibers includes two separate compositions (A) and (B). The composition (B) is an oxidative composition that includes from about 50-96% by weight water, from about 0.5-20% by weight hydrogen peroxide, at least one linear saturated 1-alkanol with 12-30 carbon atoms, at least one mono- or diester of ethylene glycol with palmitic acid and/or stearic acid as well as mixtures thereof, at least one ethoxylated fatty alcohol of the formula (III)

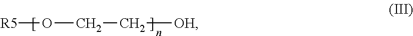

where R5 stands for a saturated or unsaturated, branched or unbranched C₈-C₂₄ alkyl group and n stands for an integer from 10 to 30, at least one ethoxylated fatty alcohol of formula (IV)

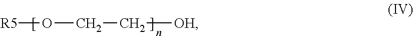

where R5 stands for a saturated or unsaturated, branched or unbranched C₈-C₂₄ alkyl group and n stands for an integer from about 50 to about 150, at least one polyol selected from C₂-C₉ alkanols with 2-6 hydroxyl groups, polyethylene glycols with 3-20 ethylene oxide units, or mixtures thereof, and at least one oil, wherein all the quantitative amounts are based on the weight of the oxidative composition. The composition (A) is present in the form of a soap-based gel and includes from about 25-85% by weight water, ammonia, from about 5-20% by weight of at least one salt of a C₁₂-C₂₂ fatty acid, from about 2-20% by weight of at least one polyethylene glycol ether of a linear saturated or unsaturated C₁₀-C₁₈ alkanol with 1-5 ethylene oxide units in the molecule, from about 0-1% by weight of at least one oil, optionally at least one organic solvent, selected from monovalent to tetravalent C₂-C₆ alcohols, optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule and optionally at least one oxidative dye precursor, wherein composition (A) has a pH in the range of from about 8 to about 11.5 measured at 20° C., wherein all the quantitative amounts are based on the weight of the composition (A).

In another embodiment, a method for oxidative color change of keratinic fibers includes providing an oxidative composition (B) that includes from about 50 to about 96% by weight water, from about 0.5 to about 20% by weight hydrogen peroxide, at least one linear saturated 1-alkanol with 12 to 30 carbon atoms, at least one mono- or diester of ethylene glycol with palmitic acid and/or stearic acid as well as mixtures thereof, at least one ethoxylated fatty alcohol of the formula (III)

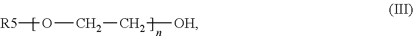

where R5 stands for a branched or unbranched, saturated or unsaturated C₈-C₂₄ alkyl group and n stands for an integer from 10 to 30, at least one ethoxylated fatty alcohol of formula (IV)

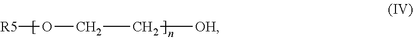

where R5 stands for a branched or unbranched, saturated or unsaturated C₈-C₂₄ alkyl group and n stands for an integer from 50 to 150, at least one polyol selected from C₂-C₉ alkanols with 2-6 hydroxyl groups, polyethylene glycols with 3-20 ethylene oxide units, or mixtures thereof, and at least one oil, wherein all the quantitative amounts are based on the weight of the oxidative composition. A composition (A) is provided, which is in the form of a soap-based gel and includes from about 25-85% by weight water, ammonia, from about 5-20% by weight of at least one salt of a C₁₂-C₂₂ fatty acid, from about 2-20% by weight of at least one polyethylene glycol ether of a linear saturated or unsaturated C₁₀-C₁₈ alkanol with 1 to 5 ethylene oxide units in the molecule, a total of from about 0-1% by weight of at least one oil, at least one organic solvent, selected from monovalent to tetravalent C₂-C₆ alcohols, optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule and optionally at least one oxidative dye precursor, wherein composition (A) has a pH in the range of from about 8 to about 11.5 measured at 20° C., wherein all the quantitative amounts are based on the weight of the composition (A). A mixture of the aforementioned composition (B) and the aforementioned composition (A) is prepared. Immediately after preparing the mixture of composition (B) and composition (A), the ready-to-use agent is distributed on the fibers. The agent is left on the fibers for a period of from about 1 to about 60 minutes, then immediately thereafter the remaining agent is rinsed out of the fibers and optionally drying the fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure is to provide an improved oxidizing agent preparation for oxidative color changing agents, which are in the form of a soap-based gel and contain ammonia as an alkalizing agent and optionally at least one oxidative dye precursor and have a pH in the range of from about 8 to about 11.5, measured at 20° C., so that they can be used to prepare homogenous application mixtures having a stable viscosity and have a reduced release of ammonia during the entire treatment time.

It has surprisingly been found that aqueous hydrogen peroxide preparations containing selected 1-alkanols, ethylene glycol fatty acid esters, ethoxylated fatty alcohols, polyols and oils achieve the object as formulated in a very good manner.

A first subject matter of the present disclosure is therefore an oxidative composition for oxidative treatment of hair, comprising:

from about 50 to about 96% by weight, preferably from about 70 to about 93% by weight, especially preferably from about 80 to about 90% by weight water,

from about 0.5 to about 20% by weight hydrogen peroxide,

at least one linear saturated 1-alkanol with 12 to 30 carbon atoms, preferably in a total amount of from about 2 to about 8% by weight, especially preferably from about 3 to about 6.5% by weight,

at least one mono- or diester of ethylene glycol with palmitic acid and/or stearic acid as well as mixtures thereof,

wherein the at least one ethylene glycol fatty acid ester is preferably present in a total amount of from about 0.01-1% by weight, especially preferably from about 0.1 to about 0.8% by weight,

at least one ethoxylated fatty alcohol of formula (III)

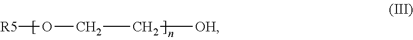

where R5 stands for a branched or unbranched, saturated or unsaturated C₁₂-C₂₈ alkyl group, preferably for a saturated, unbranched C₁₆ or C₁₈ alkyl group, and
n stands for an integer from 10 to 30, preferably for an integer from 12 to 25, more preferably for an integer from 15 to 20,
wherein the at least one ethoxylated fatty alcohol of formula (III) is preferably present in a total amount of 0.25-2% by weight, especially preferably from about 0.5 to about 1.5% by weight,

at least one ethoxylated fatty alcohol of formula (IV)

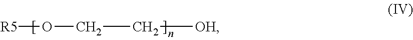

where R5 stands for a branched or unbranched, saturated or unsaturated C₁₂-C₂₈ alkyl group, preferably for a saturated unbranched C₁₆ or C₁₈ alkyl group, and
n stands for an integer from about 50 to about 150, preferably for an integer from about 80 to about 120, more preferably for the integer 100,
wherein the at least one ethoxylated fatty alcohol of formula (IV) is preferably present in a total amount of from about 0.03-0.3% by weight, especially preferably from about 0.08 to about 0.25% by weight, extremely preferably from about 0.1-0.2% by weight,

at least one polyol selected from C₂-C₉ alkanols with 2-6 hydroxyl groups and polyethylene glycols with 3-20 ethylene oxide units and mixtures thereof, preferably in a total amount of from about 1-10% by weight, especially preferably from about 2-8% by weight, extremely preferably from about 3-6% by weight,

at least one oil, preferably in a total amount of from about 0.1-5% by weight, especially preferably from about 0.2-3% by weight, extremely preferably from about 0.5-2.5% by weight,

wherein all the quantitative amounts are based on the weight of the oxidative composition.
The oxidative composition as contemplated herein contains, each based on weight, from about 50-96% by weight, preferably from about 70-93% by weight, especially preferably from about 80-90% by weight water.

The oxidative composition as contemplated herein contains, each based on weight, from about 0.5-20% by weight, preferably from about 3-12% by weight, especially preferably from about 6-9% by weight hydrogen peroxide.

Additional oxidative compositions preferred as contemplated herein are exemplified in that at least one linear saturated 1-alkanol having 12-30 carbon atoms is contained therein, preferably in a total amount of from about 2-8% by weight, especially preferably from about 3 to about 6.5% by weight, each based on the weight of the oxidation composition.

The at least one linear saturated 1-alkanol having 12 to 30 carbon atoms is preferably selected from lauryl alcohol, myristyl alcohol, cetyl alcohol, stearyl alcohol, arachidyl alcohol and behenyl alcohol as well as mixtures of these alkanols, especially preferably from cetyl alcohol, stearyl alcohol, arachidyl alcohol, behenyl alcohol and cetyl alcohol/stearyl alcohol mixtures.

Especially preferred oxidative compositions contain, each based on weight, at least one linear saturated 1-alkanol having 12 to 30 carbon atoms in a total amount of from about 2.7 to about 6% by weight, preferably in a total amount of from about 3.0 to about 5.0% by weight, wherein at least one 1-alkanol selected from cetyl alcohol, stearyl alcohol and cetyl alcohol/stearyl alcohol mixtures is contained therein.

As the fourth essential formulation ingredient, the oxidative compositions as contemplated herein contain at least one mono- or diester of ethylene glycol with palmitic acid and/or stearic acid as well as mixtures thereof, wherein the at least one ethylene glycol fatty acid ester is preferably present in a total amount of from about 0.01 to about 1% by weight, especially preferably from about 0.1 to about 0.8% by weight, based on the weight of the oxidative composition.

Additional oxidative compositions that are especially preferred as contemplated herein are exemplified in that they contain at least one compound selected from ethylene glycol monostearate, ethylene glycol distearate, ethylene glycol monopalmitate and ethylene glycol dipalmitate as well as mixtures thereof as the ethylene glycol fatty acid ester.

The ethylene glycol fatty acid esters in combination with the at least one long chain 1-alkanol result in a reduction in the ammonia odor. The odor reduction effect is especially pronounced when the ethylene glycol fatty acid ester and the at least one long chain 1-alkanol are present in certain ratios relative to one another. Therefore, the ethylene glycol fatty acid esters are also preferably present in certain amounts in the oxidative composition as contemplated herein.

It is especially preferred if the oxidative composition as contemplated herein contains, based on its weight, one or more ethylene glycol fatty acid esters in a total amount of from about 0.01 to about 1% by weight, especially preferably from about 0.1 to about 0.8% by weight.

In another especially preferred embodiment, an oxidative composition as contemplated herein is exemplified in that it contains as the ethylene glycol fatty acid ester at least one compound selected from ethylene glycol monostearate, ethylene glycol distearate, ethylene glycol monopalmitate and ethylene glycol dipalmitate as well as mixtures thereof in a total amount of from about 0.01 to about 1% by weight, preferably from about 0.1 to about 0.8% by weight, based on the weight of the oxidative composition as contemplated herein.

As the fifth essential formulation ingredient, the oxidative compositions as contemplated herein contain at least one ethoxylated fatty alcohol of formula (III)

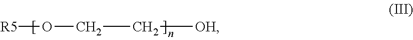

where R5 stands for a branched or unbranched, saturated or unsaturated C₈-C₂₄ alkyl group, preferably for a saturated, unbranched C₁₆ or C₁₈ alkyl group and n stands for an integer from 10 to 30, preferably for an integer from 12 to 25, more preferably for an integer from 15 to 20, wherein the at least one ethoxylated fatty alcohol of formula (III) is preferably present in a total amount of 0.25-2% by weight, especially preferably from about 0.5 to about 1.5% by weight.

It is most especially preferred if one or more ethoxylated fatty alcohols from the following group are used as the ethoxylated fatty alcohol of formula (III):

decan-1-ol (decyl alcohol) ethoxylated with 10 EO,

decan-1-ol (decyl alcohol) ethoxylated with 11 EO,

decan-1-ol (decyl alcohol) ethoxylated with 12 EO,

decan-1-ol (decyl alcohol) ethoxylated with 13 EO,

decan-1-ol (decyl alcohol) ethoxylated with 14 EO,

decan-1-ol (decyl alcohol) ethoxylated with 15 EO,

decan-1-ol (decyl alcohol) ethoxylated with 16 EO,

decan-1-ol (decyl alcohol) ethoxylated with 17 EO,

decan-1-ol (decyl alcohol) ethoxylated with 18 EO,

decan-1-ol (decyl alcohol) ethoxylated with 19 EO,

decan-1-ol (decyl alcohol) ethoxylated with 20 EO,

decan-1-ol (decyl alcohol) ethoxylated with 21 EO,

decan-1-ol (decyl alcohol) ethoxylated with 22 EO,

decan-1-ol (decyl alcohol) ethoxylated with 23 EO,

decan-1-ol (decyl alcohol) ethoxylated with 24 EO,

decan-1-ol (decyl alcohol) ethoxylated with 25 EO,

decan-1-ol (decyl alcohol) ethoxylated with 26 EO,

decan-1-ol (decyl alcohol) ethoxylated with 27 EO,

decan-1-ol (decyl alcohol) ethoxylated with 28 EO,

decan-1-ol (decyl alcohol) ethoxylated with 29 EO,

decan-1-ol (decyl alcohol) ethoxylated with 30 EO,

dodecan-1-ol (dodecyl alcohol, lauryl alcohol) ethoxylated with 10 EO,

dodecan-1-ol (dodecyl alcohol, lauryl alcohol) ethoxylated with 11 EO,

dodecan-1-ol (dodecyl alcohol, lauryl alcohol) ethoxylated with 12 EO,

dodecan-1-ol (dodecyl alcohol, lauryl alcohol) ethoxylated with 13 EO,

dodecan-1-ol (dodecyl alcohol, lauryl alcohol) ethoxylated with 14 EO,

dodecan-1-ol (dodecyl alcohol, lauryl alcohol) ethoxylated with 15 EO,

dodecan-1-ol (dodecyl alcohol, lauryl alcohol) ethoxylated with 16 EO,

dodecan-1-ol (dodecyl alcohol, lauryl alcohol) ethoxylated with 17 EO,

dodecan-1-ol (dodecyl alcohol, lauryl alcohol) ethoxylated with 18 EO,

dodecan-1-ol (dodecyl alcohol, lauryl alcohol) ethoxylated with 19 EO,

dodecan-1-ol (dodecyl alcohol, lauryl alcohol) ethoxylated with 20 EO,

dodecan-1-ol (dodecyl alcohol, lauryl alcohol) ethoxylated with 21 EO,

dodecan-1-ol (dodecyl alcohol, lauryl alcohol) ethoxylated with 22 EO,

dodecan-1-ol (dodecyl alcohol, lauryl alcohol) ethoxylated with 23 EO,

dodecan-1-ol (dodecyl alcohol, lauryl alcohol) ethoxylated with 24 EO,

dodecan-1-ol (dodecyl alcohol, lauryl alcohol) ethoxylated with 25 EO,

dodecan-1-ol (dodecyl alcohol, lauryl alcohol) ethoxylated with 26 EO,

dodecan-1-ol (dodecyl alcohol, lauryl alcohol) ethoxylated with 27 EO,

dodecan-1-ol (dodecyl alcohol, lauryl alcohol) ethoxylated with 28 EO,

dodecan-1-ol (dodecyl alcohol, lauryl alcohol) ethoxylated with 29 EO,

dodecan-1-ol (dodecyl alcohol, lauryl alcohol) ethoxylated with 30 EO,

tetradecan-1-ol, (tetradecyl alcohol, myristyl alcohol) ethoxylated with 10 EO,

tetradecan-1-ol, (tetradecyl alcohol, myristyl alcohol) ethoxylated with 11 EO,

tetradecan-1-ol, (tetradecyl alcohol, myristyl alcohol) ethoxylated with 12 EO,

tetradecan-1-ol, (tetradecyl alcohol, myristyl alcohol) ethoxylated with 13 EO,

tetradecan-1-ol, (tetradecyl alcohol, myristyl alcohol) ethoxylated with 14 EO,

tetradecan-1-ol, (tetradecyl alcohol, myristyl alcohol) ethoxylated with 15 EO,

tetradecan-1-ol, (tetradecyl alcohol, myristyl alcohol) ethoxylated with 16 EO,

tetradecan-1-ol, (tetradecyl alcohol, myristyl alcohol) ethoxylated with 17 EO,

tetradecan-1-ol, (tetradecyl alcohol, myristyl alcohol) ethoxylated with 18 EO,

tetradecan-1-ol, (tetradecyl alcohol, myristyl alcohol) ethoxylated with 19 EO,

tetradecan-1-ol, (tetradecyl alcohol, myristyl alcohol) ethoxylated with 20 EO,

tetradecan-1-ol, (tetradecyl alcohol, myristyl alcohol) ethoxylated with 21 EO,

tetradecan-1-ol, (tetradecyl alcohol, myristyl alcohol) ethoxylated with 22 EO,

tetradecan-1-ol, (tetradecyl alcohol, myristyl alcohol) ethoxylated with 23 EO,

tetradecan-1-ol, (tetradecyl alcohol, myristyl alcohol) ethoxylated with 24 EO,

tetradecan-1-ol, (tetradecyl alcohol, myristyl alcohol) ethoxylated with 25 EO,

tetradecan-1-ol, (tetradecyl alcohol, myristyl alcohol) ethoxylated with 26 EO,

tetradecan-1-ol, (tetradecyl alcohol, myristyl alcohol) ethoxylated with 27 EO,

tetradecan-1-ol, (tetradecyl alcohol, myristyl alcohol) ethoxylated with 28 EO,

tetradecan-1-ol, (tetradecyl alcohol, myristyl alcohol) ethoxylated with 29 EO,

tetradecan-1-ol, (tetradecyl alcohol, myristyl alcohol) ethoxylated with 30 EO,

hexadecan-1-ol (hexadecyl alcohol, cetyl alcohol, palmityl alcohol) ethoxylated with 10 EO,

hexadecan-1-ol (hexadecyl alcohol, cetyl alcohol, palmityl alcohol) ethoxylated with 11 EO,

hexadecan-1-ol (hexadecyl alcohol, cetyl alcohol, palmityl alcohol) ethoxylated with 12 EO,

hexadecan-1-ol (hexadecyl alcohol, cetyl alcohol, palmityl alcohol) ethoxylated with 13 EO,

hexadecan-1-ol (hexadecyl alcohol, cetyl alcohol, palmityl alcohol) ethoxylated with 14 EO,

hexadecan-1-ol (hexadecyl alcohol, cetyl alcohol, palmityl alcohol) ethoxylated with 15 EO,

hexadecan-1-ol (hexadecyl alcohol, cetyl alcohol, palmityl alcohol) ethoxylated with 16 EO,

hexadecan-1-ol (hexadecyl alcohol, cetyl alcohol, palmityl alcohol) ethoxylated with 17 EO,

hexadecan-1-ol (hexadecyl alcohol, cetyl alcohol, palmityl alcohol) ethoxylated with 18 EO,

hexadecan-1-ol (hexadecyl alcohol, cetyl alcohol, palmityl alcohol) ethoxylated with 19 EO,

hexadecan-1-ol (hexadecyl alcohol, cetyl alcohol, palmityl alcohol) ethoxylated with 20 EO,

hexadecan-1-ol (hexadecyl alcohol, cetyl alcohol, palmityl alcohol) ethoxylated with 21 EO,

hexadecan-1-ol (hexadecyl alcohol, cetyl alcohol, palmityl alcohol) ethoxylated with 22 EO,

hexadecan-1-ol (hexadecyl alcohol, cetyl alcohol, palmityl alcohol) ethoxylated with 23 EO,

hexadecan-1-ol (hexadecyl alcohol, cetyl alcohol, palmityl alcohol) ethoxylated with 24 EO,

hexadecan-1-ol (hexadecyl alcohol, cetyl alcohol, palmityl alcohol) ethoxylated with 25 EO,

hexadecan-1-ol (hexadecyl alcohol, cetyl alcohol, palmityl alcohol) ethoxylated with 26 EO,

hexadecan-1-ol (hexadecyl alcohol, cetyl alcohol, palmityl alcohol) ethoxylated with 27 EO,

hexadecan-1-ol (hexadecyl alcohol, cetyl alcohol, palmityl alcohol) ethoxylated with 28 EO,

hexadecan-1-ol (hexadecyl alcohol, cetyl alcohol, palmityl alcohol) ethoxylated with 29 EO,

hexadecan-1-ol (hexadecyl alcohol, cetyl alcohol, palmityl alcohol) ethoxylated with 30 EO,

octadecan-1-ol (octadecyl alcohol, stearyl alcohol) ethoxylated with 10 EO,

octadecan-1-ol (octadecyl alcohol, stearyl alcohol) ethoxylated with 11 EO,

octadecan-1-ol (octadecyl alcohol, stearyl alcohol) ethoxylated with 12 EO,

octadecan-1-ol (octadecyl alcohol, stearyl alcohol) ethoxylated with 13 EO,

octadecan-1-ol (octadecyl alcohol, stearyl alcohol) ethoxylated with 14 EO,

octadecan-1-ol (octadecyl alcohol, stearyl alcohol) ethoxylated with 15 EO,

octadecan-1-ol (octadecyl alcohol, stearyl alcohol) ethoxylated with 16 EO,

octadecan-1-ol (octadecyl alcohol, stearyl alcohol) ethoxylated with 17 EO,

octadecan-1-ol (octadecyl alcohol, stearyl alcohol) ethoxylated with 18 EO,

octadecan-1-ol (octadecyl alcohol, stearyl alcohol) ethoxylated with 19 EO,

octadecan-1-ol (octadecyl alcohol, stearyl alcohol) ethoxylated with 20 EO,

octadecan-1-ol (octadecyl alcohol, stearyl alcohol) ethoxylated with 21 EO,

octadecan-1-ol (octadecyl alcohol, stearyl alcohol) ethoxylated with 22 EO,

octadecan-1-ol (octadecyl alcohol, stearyl alcohol) ethoxylated with 23 EO,

octadecan-1-ol (octadecyl alcohol, stearyl alcohol) ethoxylated with 24 EO,

octadecan-1-ol (octadecyl alcohol, stearyl alcohol) ethoxylated with 25 EO,

octadecan-1-ol (octadecyl alcohol, stearyl alcohol) ethoxylated with 26 EO,

octadecan-1-ol (octadecyl alcohol, stearyl alcohol) ethoxylated with 27 EO,

octadecan-1-ol (octadecyl alcohol, stearyl alcohol) ethoxylated with 28 EO,

octadecan-1-ol (octadecyl alcohol, stearyl alcohol) ethoxylated with 29 EO,

octadecan-1-ol (octadecyl alcohol, stearyl alcohol) ethoxylated with 30 EO,

(9Z)-octadec-9-en-1-ol (oleyl alcohol) ethoxylated with 10 EO,

(9Z)-octadec-9-en-1-ol (oleyl alcohol) ethoxylated with 11 EO,

(9Z)-octadec-9-en-1-ol (oleyl alcohol) ethoxylated with 12 EO,

(9Z)-octadec-9-en-1-ol (oleyl alcohol) ethoxylated with 13 EO,

(9Z)-octadec-9-en-1-ol (oleyl alcohol) ethoxylated with 14 EO,

(9Z)-octadec-9-en-1-ol (oleyl alcohol) ethoxylated with 15 EO,

(9Z)-octadec-9-en-1-ol (oleyl alcohol) ethoxylated with 16 EO,

(9Z)-octadec-9-en-1-ol (oleyl alcohol) ethoxylated with 17 EO,

(9Z)-octadec-9-en-1-ol (oleyl alcohol) ethoxylated with 18 EO,

(9Z)-octadec-9-en-1-ol (oleyl alcohol) ethoxylated with 19 EO,

(9Z)-octadec-9-en-1-ol (oleyl alcohol) ethoxylated with 20 EO,

(9Z)-octadec-9-en-1-ol (oleyl alcohol) ethoxylated with 21 EO,

(9Z)-octadec-9-en-1-ol (oleyl alcohol) ethoxylated with 22 EO,

(9Z)-octadec-9-en-1-ol (oleyl alcohol) ethoxylated with 23 EO,

(9Z)-octadec-9-en-1-ol (oleyl alcohol) ethoxylated with 24 EO,

(9Z)-octadec-9-en-1-ol (oleyl alcohol) ethoxylated with 25 EO,

(9Z)-octadec-9-en-1-ol (oleyl alcohol) ethoxylated with 26 EO,

(9Z)-octadec-9-en-1-ol (oleyl alcohol) ethoxylated with 27 EO,

(9Z)-octadec-9-en-1-ol (oleyl alcohol) ethoxylated with 28 EO,

(9Z)-octadec-9-en-1-ol (oleyl alcohol) ethoxylated with 29 EO,

(9Z)-octadec-9-en-1-ol (oleyl alcohol) ethoxylated with 30 EO,

(9E)-octadec-9-en-1-ol (elaidyl alcohol) ethoxylated with 10 EO,

(9E)-octadec-9-en-1-ol (elaidyl alcohol) ethoxylated with 11 EO,

(9E)-octadec-9-en-1-ol (elaidyl alcohol) ethoxylated with 12 EO,

(9E)-octadec-9-en-1-ol (elaidyl alcohol) ethoxylated with 13 EO,

(9E)-octadec-9-en-1-ol (elaidyl alcohol) ethoxylated with 14 EO,

(9E)-octadec-9-en-1-ol (elaidyl alcohol) ethoxylated with 15 EO,

(9E)-octadec-9-en-1-ol (elaidyl alcohol) ethoxylated with 16 EO,

(9E)-octadec-9-en-1-ol (elaidyl alcohol) ethoxylated with 17 EO,

(9E)-octadec-9-en-1-ol (elaidyl alcohol) ethoxylated with 18 EO,

(9E)-octadec-9-en-1-ol (elaidyl alcohol) ethoxylated with 19 EO,

(9E)-octadec-9-en-1-ol (elaidyl alcohol) ethoxylated with 20 EO,

(9E)-octadec-9-en-1-ol (elaidyl alcohol) ethoxylated with 21 EO,

(9E)-octadec-9-en-1-ol (elaidyl alcohol) ethoxylated with 22 EO,

(9E)-octadec-9-en-1-ol (elaidyl alcohol) ethoxylated with 23 EO,

(9E)-octadec-9-en-1-ol (elaidyl alcohol) ethoxylated with 24 EO,

(9E)-octadec-9-en-1-ol (elaidyl alcohol) ethoxylated with 25 EO,

(9E)-octadec-9-en-1-ol (elaidyl alcohol) ethoxylated with 26 EO,

(9E)-octadec-9-en-1-ol (elaidyl alcohol) ethoxylated with 27 EO,

(9E)-octadec-9-en-1-ol (elaidyl alcohol) ethoxylated with 28 EO,

(9E)-octadec-9-en-1-ol (elaidyl alcohol) ethoxylated with 29 EO,

(9E)-octadec-9-en-1-ol (elaidyl alcohol) ethoxylated with 30 EO,

(9Z,12Z)-octadeca-9,12-dien-1-ol (linoleyl alcohol) ethoxylated with 10 EO,

(9Z,12Z)-octadeca-9,12-dien-1-ol (linoleyl alcohol) ethoxylated with 11 EO,

(9Z,12Z)-octadeca-9,12-dien-1-ol (linoleyl alcohol) ethoxylated with 12 EO,

(9Z,12Z)-octadeca-9,12-dien-1-ol (linoleyl alcohol) ethoxylated with 13 EO,

(9Z,12Z)-octadeca-9,12-dien-1-ol (linoleyl alcohol) ethoxylated with 14 EO,

(9Z,12Z)-octadeca-9,12-dien-1-ol (linoleyl alcohol) ethoxylated with 15 EO,

(9Z,12Z)-octadeca-9,12-dien-1-ol (linoleyl alcohol) ethoxylated with 16 EO,

(9Z,12Z)-octadeca-9,12-dien-1-ol (linoleyl alcohol) ethoxylated with 17 EO,

(9Z,12Z)-octadeca-9,12-dien-1-ol (linoleyl alcohol) ethoxylated with 18 EO,

(9Z,12Z)-octadeca-9,12-dien-1-ol (linoleyl alcohol) ethoxylated with 19 EO,

(9Z,12Z)-octadeca-9,12-dien-1-ol (linoleyl alcohol) ethoxylated with 20 EO,

(9Z,12Z)-octadeca-9,12-dien-1-ol (linoleyl alcohol) ethoxylated with 21 EO,

(9Z,12Z)-octadeca-9,12-dien-1-ol (linoleyl alcohol) ethoxylated with 22 EO,

(9Z,12Z)-octadeca-9,12-dien-1-ol (linoleyl alcohol) ethoxylated with 23 EO,

(9Z,12Z)-octadeca-9,12-dien-1-ol (linoleyl alcohol) ethoxylated with 24 EO,

(9Z,12Z)-octadeca-9,12-dien-1-ol (linoleyl alcohol) ethoxylated with 25 EO,

(9Z,12Z)-octadeca-9,12-dien-1-ol (linoleyl alcohol) ethoxylated with 26 EO,

(9Z,12Z)-octadeca-9,12-dien-1-ol (linoleyl alcohol) ethoxylated with 27 EO,

(9Z,12Z)-octadeca-9,12-dien-1-ol (linoleyl alcohol) ethoxylated with 28 EO,

(9Z,12Z)-octadeca-9,12-dien-1-ol (linoleyl alcohol) ethoxylated with 29 EO,

(9Z,12Z)-octadeca-9,12-dien-1-ol (linoleyl alcohol) ethoxylated with 30 EO,

eicosan-1-ol (eicosyl alcohol, arachyl alcohol) ethoxylated with 10 EO,

eicosan-1-ol (eicosyl alcohol, arachyl alcohol) ethoxylated with 11 EO,

eicosan-1-ol (eicosyl alcohol, arachyl alcohol) ethoxylated with 12 EO,

eicosan-1-ol (eicosyl alcohol, arachyl alcohol) ethoxylated with 13 EO,

eicosan-1-ol (eicosyl alcohol, arachyl alcohol) ethoxylated with 14 EO,

eicosan-1-ol (eicosyl alcohol, arachyl alcohol) ethoxylated with 15 EO,

eicosan-1-ol (eicosyl alcohol, arachyl alcohol) ethoxylated with 16 EO,

eicosan-1-ol (eicosyl alcohol, arachyl alcohol) ethoxylated with 17 EO,

eicosan-1-ol (eicosyl alcohol, arachyl alcohol) ethoxylated with 18 EO,

eicosan-1-ol (eicosyl alcohol, arachyl alcohol) ethoxylated with 19 EO,

eicosan-1-ol (eicosyl alcohol, arachyl alcohol) ethoxylated with 20 EO,

eicosan-1-ol (eicosyl alcohol, arachyl alcohol) ethoxylated with 21 EO,

eicosan-1-ol (eicosyl alcohol, arachyl alcohol) ethoxylated with 22 EO,

eicosan-1-ol (eicosyl alcohol, arachyl alcohol) ethoxylated with 23 EO,

eicosan-1-ol (eicosyl alcohol, arachyl alcohol) ethoxylated with 24 EO,

eicosan-1-ol (eicosyl alcohol, arachyl alcohol) ethoxylated with 25 EO,

eicosan-1-ol (eicosyl alcohol, arachyl alcohol) ethoxylated with 26 EO,

eicosan-1-ol (eicosyl alcohol, arachyl alcohol) ethoxylated with 27 EO,

eicosan-1-ol (eicosyl alcohol, arachyl alcohol) ethoxylated with 28 EO,

eicosan-1-ol (eicosyl alcohol, arachyl alcohol) ethoxylated with 29 EO,

eicosan-1-ol (eicosyl alcohol, arachyl alcohol) ethoxylated with 30 EO,

docosan-1-ol (docosyl alcohol, behenyl alcohol) ethoxylated with 10 EO,

docosan-1-ol (docosyl alcohol, behenyl alcohol) ethoxylated with 11 EO,

docosan-1-ol (docosyl alcohol, behenyl alcohol) ethoxylated with 12 EO,

docosan-1-ol (docosyl alcohol, behenyl alcohol) ethoxylated with 13 EO,

docosan-1-ol (docosyl alcohol, behenyl alcohol) ethoxylated with 14 EO,

docosan-1-ol (docosyl alcohol, behenyl alcohol) ethoxylated with 15 EO,

docosan-1-ol (docosyl alcohol, behenyl alcohol) ethoxylated with 16 EO,

docosan-1-ol (docosyl alcohol, behenyl alcohol) ethoxylated with 17 EO,

docosan-1-ol (docosyl alcohol, behenyl alcohol) ethoxylated with 18 EO,

docosan-1-ol (docosyl alcohol, behenyl alcohol) ethoxylated with 19 EO,

docosan-1-ol (docosyl alcohol, behenyl alcohol) ethoxylated with 20 EO,

docosan-1-ol (docosyl alcohol, behenyl alcohol) ethoxylated with 21 EO,

docosan-1-ol (docosyl alcohol, behenyl alcohol) ethoxylated with 22 EO,

docosan-1-ol (docosyl alcohol, behenyl alcohol) ethoxylated with 23 EO,

docosan-1-ol (docosyl alcohol, behenyl alcohol) ethoxylated with 24 EO,

docosan-1-ol (docosyl alcohol, behenyl alcohol) ethoxylated with 25 EO,

docosan-1-ol (docosyl alcohol, behenyl alcohol) ethoxylated with 26 EO,

docosan-1-ol (docosyl alcohol, behenyl alcohol) ethoxylated with 27 EO,

docosan-1-ol (docosyl alcohol, behenyl alcohol) ethoxylated with 28 EO,

docosan-1-ol (docosyl alcohol, behenyl alcohol) ethoxylated with 29 EO,

docosan-1-ol (docosyl alcohol, behenyl alcohol) ethoxylated with 30 EO as well as mixtures thereof.

Extremely preferred are

decan-1-ol (decyl alcohol) ethoxylated with 10 EO,

decan-1-ol (decyl alcohol) ethoxylated with 20 EO,

decan-1-ol (decyl alcohol) ethoxylated with 30 EO,

dodecan-1-ol (dodecyl alcohol, lauryl alcohol) ethoxylated with 10 EO,

dodecan-1-ol (dodecyl alcohol, lauryl alcohol) ethoxylated with 20 EO,

dodecan-1-ol (dodecyl alcohol, lauryl alcohol) ethoxylated with 30 EO,

hexadecan-1-ol (hexadecyl alcohol, cetyl alcohol, palmityl alcohol) ethoxylated with 10 EO,

hexadecan-1-ol (hexadecyl alcohol, cetyl alcohol, palmityl alcohol) ethoxylated with 12 EO,

hexadecan-1-ol (hexadecyl alcohol, cetyl alcohol, palmityl alcohol) ethoxylated with 15 EO,

hexadecan-1-ol (hexadecyl alcohol, cetyl alcohol, palmityl alcohol) ethoxylated with 20 EO,

hexadecan-1-ol (hexadecyl alcohol, cetyl alcohol, palmityl alcohol) ethoxylated with 25 EO,

hexadecan-1-ol (hexadecyl alcohol, cetyl alcohol, palmityl alcohol) ethoxylated with 30 EO,

octadecan-1-ol (octadecyl alcohol, stearyl alcohol) ethoxylated with 10 EO,

octadecan-1-ol (octadecyl alcohol, stearyl alcohol) ethoxylated with 12 EO,

octadecan-1-ol (octadecyl alcohol, stearyl alcohol) ethoxylated with 15 EO,

octadecan-1-ol (octadecyl alcohol, stearyl alcohol) ethoxylated with 20 EO,

octadecan-1-ol (octadecyl alcohol, stearyl alcohol) ethoxylated with 25 EO,

octadecan-1-ol (octadecyl alcohol, stearyl alcohol) ethoxylated with 30 EO,

eicosan-1-ol (eicosyl alcohol, arachyl alcohol) ethoxylated with 10 EO,

eicosan-1-ol (eicosyl alcohol, arachyl alcohol) ethoxylated with 12 EO,

eicosan-1-ol (eicosyl alcohol, arachyl alcohol) ethoxylated with 15 EO, 18

eicosan-1-ol (eicosyl alcohol, arachyl alcohol) ethoxylated with 20 EO,

eicosan-1-ol (eicosyl alcohol, arachyl alcohol) ethoxylated with 25 EO,

eicosan-1-ol (eicosyl alcohol, arachyl alcohol) ethoxylated with 30 EO,

docosan-1-ol (docosyl alcohol, behenyl alcohol) ethoxylated with 10 EO

docosan-1-ol (docosyl alcohol, behenyl alcohol) ethoxylated with 12 EO

docosan-1-ol (docosyl alcohol, behenyl alcohol) ethoxylated with 15 EO

docosan-1-ol (docosyl alcohol, behenyl alcohol) ethoxylated with 20 EO

docosan-1-ol (docosyl alcohol, behenyl alcohol) ethoxylated with 25 EO

docosan-1-ol (docosyl alcohol, behenyl alcohol) ethoxylated with 30 EO as well as mixtures thereof.

The oxidative compositions as contemplated herein contain at least one ethoxylated fatty alcohol of formula (IV) as the sixth essential formulation ingredient:

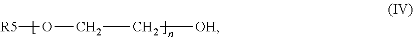

where R5 stands for a branched or unbranched, saturated or unsaturated C₈-C₂₄ alkyl group, preferably for a saturated unbranched C₁₆ or C₁₈ alkyl group and n stands for an integer from about 50 to about 150, preferably for an integer from about 80 to about 120, more preferably the number 100,
wherein the at least one ethoxylated fatty alcohol of formula (IV) is preferably present in a total amount of from about 0.03 to about 0.3% by weight, especially preferably from about 0.08 to about 0.25% by weight, extremely preferably from about 0.1 to about 0.2% by weight.

It is most especially preferred if it contains one or more ethoxylated fatty alcohols from the following group as the ethoxylated fatty alcohol

dodecan-1-ol (dodecyl alcohol, lauryl alcohol) ethoxylated with 50 EO,

dodecan-1-ol (dodecyl alcohol, lauryl alcohol) ethoxylated with 51 EO,

dodecan-1-ol (dodecyl alcohol, lauryl alcohol) ethoxylated with 52 EO,

dodecan-1-ol (dodecyl alcohol, lauryl alcohol) ethoxylated with 53 EO,

dodecan-1-ol (dodecyl alcohol, lauryl alcohol) ethoxylated with 54 EO,

dodecan-1-ol (dodecyl alcohol, lauryl alcohol) ethoxylated with 55 EO,

dodecan-1-ol (dodecyl alcohol, lauryl alcohol) ethoxylated with 56 EO,

dodecan-1-ol (dodecyl alcohol, lauryl alcohol) ethoxylated with 57 EO,

dodecan-1-ol (dodecyl alcohol, lauryl alcohol) ethoxylated with 58 EO,

dodecan-1-ol (dodecyl alcohol, lauryl alcohol) ethoxylated with 59 EO,

dodecan-1-ol (dodecyl alcohol, lauryl alcohol) ethoxylated with 60 EO,

dodecan-1-ol (dodecyl alcohol, lauryl alcohol) ethoxylated with 80 EO,

dodecan-1-ol (dodecyl alcohol, lauryl alcohol) ethoxylated with 81 EO,

dodecan-1-ol (dodecyl alcohol, lauryl alcohol) ethoxylated with 82 EO,

dodecan-1-ol (dodecyl alcohol, lauryl alcohol) ethoxylated with 83 EO,

dodecan-1-ol (dodecyl alcohol, lauryl alcohol) ethoxylated with 84 EO,

dodecan-1-ol (dodecyl alcohol, lauryl alcohol) ethoxylated with 85 EO,

dodecan-1-ol (dodecyl alcohol, lauryl alcohol) ethoxylated with 86 EO,

dodecan-1-ol (dodecyl alcohol, lauryl alcohol) ethoxylated with 87 EO,

dodecan-1-ol (dodecyl alcohol, lauryl alcohol) ethoxylated with 88 EO,

dodecan-1-ol (dodecyl alcohol, lauryl alcohol) ethoxylated with 89 EO,

dodecan-1-ol (dodecyl alcohol, lauryl alcohol) ethoxylated with 90 EO,

dodecan-1-ol (dodecyl alcohol, lauryl alcohol) ethoxylated with 91 EO,

dodecan-1-ol (dodecyl alcohol, lauryl alcohol) ethoxylated with 92 EO,

dodecan-1-ol (dodecyl alcohol, lauryl alcohol) ethoxylated with 93 EO,

dodecan-1-ol (dodecyl alcohol, lauryl alcohol) ethoxylated with 94 EO,

dodecan-1-ol (dodecyl alcohol, lauryl alcohol) ethoxylated with 95 EO,

dodecan-1-ol (dodecyl alcohol, lauryl alcohol) ethoxylated with 96 EO,

dodecan-1-ol (dodecyl alcohol, lauryl alcohol) ethoxylated with 97 EO,

dodecan-1-ol (dodecyl alcohol, lauryl alcohol) ethoxylated with 98 EO,

dodecan-1-ol (dodecyl alcohol, lauryl alcohol) ethoxylated with 99 EO,

dodecan-1-ol (dodecyl alcohol, lauryl alcohol) ethoxylated with 100 EO,

dodecan-1-ol (dodecyl alcohol, lauryl alcohol) ethoxylated with 101 EO,

dodecan-1-ol (dodecyl alcohol, lauryl alcohol) ethoxylated with 102 EO,

dodecan-1-ol (dodecyl alcohol, lauryl alcohol) ethoxylated with 103 EO,

dodecan-1-ol (dodecyl alcohol, lauryl alcohol) ethoxylated with 104 EO,

dodecan-1-ol (dodecyl alcohol, lauryl alcohol) ethoxylated with 105 EO,

dodecan-1-ol (dodecyl alcohol, lauryl alcohol) ethoxylated with 106 EO,

dodecan-1-ol (dodecyl alcohol, lauryl alcohol) ethoxylated with 107 EO,

dodecan-1-ol (dodecyl alcohol, lauryl alcohol) ethoxylated with 108 EO,

dodecan-1-ol (dodecyl alcohol, lauryl alcohol) ethoxylated with 109 EO,

dodecan-1-ol (dodecyl alcohol, lauryl alcohol) ethoxylated with 110 EO,

dodecan-1-ol (dodecyl alcohol, lauryl alcohol) ethoxylated with 111 EO,

dodecan-1-ol (dodecyl alcohol, lauryl alcohol) ethoxylated with 112 EO,

dodecan-1-ol (dodecyl alcohol, lauryl alcohol) ethoxylated with 113 EO,

dodecan-1-ol (dodecyl alcohol, lauryl alcohol) ethoxylated with 114 EO,

dodecan-1-ol (dodecyl alcohol, lauryl alcohol) ethoxylated with 115 EO,

dodecan-1-ol (dodecyl alcohol, lauryl alcohol) ethoxylated with 116 EO,

dodecan-1-ol (dodecyl alcohol, lauryl alcohol) ethoxylated with 117 EO,

dodecan-1-ol (dodecyl alcohol, lauryl alcohol) ethoxylated with 118 EO,

dodecan-1-ol (dodecyl alcohol, lauryl alcohol) ethoxylated with 119 EO,

dodecan-1-ol (dodecyl alcohol, lauryl alcohol) ethoxylated with 120 EO,

tetradecan-1-ol, (tetradecyl alcohol, myristyl alcohol) ethoxylated with 50 EO,

tetradecan-1-ol, (tetradecyl alcohol, myristyl alcohol) ethoxylated with 51 EO,

tetradecan-1-ol, (tetradecyl alcohol, myristyl alcohol) ethoxylated with 52 EO,

tetradecan-1-ol, (tetradecyl alcohol, myristyl alcohol) ethoxylated with 53 EO,

tetradecan-1-ol, (tetradecyl alcohol, myristyl alcohol) ethoxylated with 54 EO,

tetradecan-1-ol, (tetradecyl alcohol, myristyl alcohol) ethoxylated with 55 EO,

tetradecan-1-ol, (tetradecyl alcohol, myristyl alcohol) ethoxylated with 56 EO,

tetradecan-1-ol, (tetradecyl alcohol, myristyl alcohol) ethoxylated with 57 EO,

tetradecan-1-ol, (tetradecyl alcohol, myristyl alcohol) ethoxylated with 58 EO,

tetradecan-1-ol, (tetradecyl alcohol, myristyl alcohol) ethoxylated with 59 EO,

tetradecan-1-ol, (tetradecyl alcohol, myristyl alcohol) ethoxylated with 60 EO,

tetradecan-1-ol, (tetradecyl alcohol, myristyl alcohol) ethoxylated with 80 EO,

tetradecan-1-ol, (tetradecyl alcohol, myristyl alcohol) ethoxylated with 81 EO,

tetradecan-1-ol, (tetradecyl alcohol, myristyl alcohol) ethoxylated with 82 EO,

tetradecan-1-ol, (tetradecyl alcohol, myristyl alcohol) ethoxylated with 83 EO,

tetradecan-1-ol, (tetradecyl alcohol, myristyl alcohol) ethoxylated with 84 EO,

tetradecan-1-ol, (tetradecyl alcohol, myristyl alcohol) ethoxylated with 85 EO,

tetradecan-1-ol, (tetradecyl alcohol, myristyl alcohol) ethoxylated with 86 EO,

tetradecan-1-ol, (tetradecyl alcohol, myristyl alcohol) ethoxylated with 87 EO,

tetradecan-1-ol, (tetradecyl alcohol, myristyl alcohol) ethoxylated with 88 EO,

tetradecan-1-ol, (tetradecyl alcohol, myristyl alcohol) ethoxylated with 89 EO,

tetradecan-1-ol, (tetradecyl alcohol, myristyl alcohol) ethoxylated with 90 EO,

tetradecan-1-ol, (tetradecyl alcohol, myristyl alcohol) ethoxylated with 91 EO,

tetradecan-1-ol, (tetradecyl alcohol, myristyl alcohol) ethoxylated with 92 EO,

tetradecan-1-ol, (tetradecyl alcohol, myristyl alcohol) ethoxylated with 93 EO,

tetradecan-1-ol, (tetradecyl alcohol, myristyl alcohol) ethoxylated with 94 EO,

tetradecan-1-ol, (tetradecyl alcohol, myristyl alcohol) ethoxylated with 95 EO,

tetradecan-1-ol, (tetradecyl alcohol, myristyl alcohol) ethoxylated with 96 EO,

tetradecan-1-ol, (tetradecyl alcohol, myristyl alcohol) ethoxylated with 97 EO,

tetradecan-1-ol, (tetradecyl alcohol, myristyl alcohol) ethoxylated with 98 EO,

tetradecan-1-ol, (tetradecyl alcohol, myristyl alcohol) ethoxylated with 99 EO,

tetradecan-1-ol, (tetradecyl alcohol, myristyl alcohol) ethoxylated with 100 EO,

tetradecan-1-ol, (tetradecyl alcohol, myristyl alcohol) ethoxylated with 101 EO,

tetradecan-1-ol, (tetradecyl alcohol, myristyl alcohol) ethoxylated with 102 EO,

tetradecan-1-ol, (tetradecyl alcohol, myristyl alcohol) ethoxylated with 103 EO,

tetradecan-1-ol, (tetradecyl alcohol, myristyl alcohol) ethoxylated with 104 EO,

tetradecan-1-ol, (tetradecyl alcohol, myristyl alcohol) ethoxylated with 105 EO,

tetradecan-1-ol, (tetradecyl alcohol, myristyl alcohol) ethoxylated with 106 EO,

tetradecan-1-ol, (tetradecyl alcohol, myristyl alcohol) ethoxylated with 107 EO,

tetradecan-1-ol, (tetradecyl alcohol, myristyl alcohol) ethoxylated with 108 EO,

tetradecan-1-ol, (tetradecyl alcohol, myristyl alcohol) ethoxylated with 109 EO,

tetradecan-1-ol, (tetradecyl alcohol, myristyl alcohol) ethoxylated with 110 EO,

tetradecan-1-ol, (tetradecyl alcohol, myristyl alcohol) ethoxylated with 111 EO,

tetradecan-1-ol, (tetradecyl alcohol, myristyl alcohol) ethoxylated with 112 EO,

tetradecan-1-ol, (tetradecyl alcohol, myristyl alcohol) ethoxylated with 113 EO,

tetradecan-1-ol, (tetradecyl alcohol, myristyl alcohol) ethoxylated with 114 EO,

tetradecan-1-ol, (tetradecyl alcohol, myristyl alcohol) ethoxylated with 115 EO,

tetradecan-1-ol, (tetradecyl alcohol, myristyl alcohol) ethoxylated with 116 EO,

tetradecan-1-ol, (tetradecyl alcohol, myristyl alcohol) ethoxylated with 117 EO,

tetradecan-1-ol, (tetradecyl alcohol, myristyl alcohol) ethoxylated with 118 EO,

tetradecan-1-ol, (tetradecyl alcohol, myristyl alcohol) ethoxylated with 119 EO,

tetradecan-1-ol, (tetradecyl alcohol, myristyl alcohol) ethoxylated with 120 EO,

hexadecan-1-ol (hexadecyl alcohol, cetyl alcohol, palmityl alcohol) ethoxylated with 50 EO,

hexadecan-1-ol (hexadecyl alcohol, cetyl alcohol, palmityl alcohol) ethoxylated with 51 EO,

hexadecan-1-ol (hexadecyl alcohol, cetyl alcohol, palmityl alcohol) ethoxylated with 52 EO,

hexadecan-1-ol (hexadecyl alcohol, cetyl alcohol, palmityl alcohol) ethoxylated with 53 EO,

hexadecan-1-ol (hexadecyl alcohol, cetyl alcohol, palmityl alcohol) ethoxylated with 54 EO,

hexadecan-1-ol (hexadecyl alcohol, cetyl alcohol, palmityl alcohol) ethoxylated with 55 EO,

hexadecan-1-ol (hexadecyl alcohol, cetyl alcohol, palmityl alcohol) ethoxylated with 56 EO,

hexadecan-1-ol (hexadecyl alcohol, cetyl alcohol, palmityl alcohol) ethoxylated with 57 EO,

hexadecan-1-ol (hexadecyl alcohol, cetyl alcohol, palmityl alcohol) ethoxylated with 58 EO,

hexadecan-1-ol (hexadecyl alcohol, cetyl alcohol, palmityl alcohol) ethoxylated with 59 EO,

hexadecan-1-ol (hexadecyl alcohol, cetyl alcohol, palmityl alcohol) ethoxylated with 60 EO,

hexadecan-1-ol (hexadecyl alcohol, cetyl alcohol, palmityl alcohol) ethoxylated with 80 EO,

hexadecan-1-ol (hexadecyl alcohol, cetyl alcohol, palmityl alcohol) ethoxylated with 81 EO,

hexadecan-1-ol (hexadecyl alcohol, cetyl alcohol, palmityl alcohol) ethoxylated with 82 EO,

hexadecan-1-ol (hexadecyl alcohol, cetyl alcohol, palmityl alcohol) ethoxylated with 83 EO,

hexadecan-1-ol (hexadecyl alcohol, cetyl alcohol, palmityl alcohol) ethoxylated with 84 EO,

hexadecan-1-ol (hexadecyl alcohol, cetyl alcohol, palmityl alcohol) ethoxylated with 85 EO,

hexadecan-1-ol (hexadecyl alcohol, cetyl alcohol, palmityl alcohol) ethoxylated with 86 EO,

hexadecan-1-ol (hexadecyl alcohol, cetyl alcohol, palmityl alcohol) ethoxylated with 87 EO,

hexadecan-1-ol (hexadecyl alcohol, cetyl alcohol, palmityl alcohol) ethoxylated with 88 EO,

hexadecan-1-ol (hexadecyl alcohol, cetyl alcohol, palmityl alcohol) ethoxylated with 89 EO,

hexadecan-1-ol (hexadecyl alcohol, cetyl alcohol, palmityl alcohol) ethoxylated with 90 EO,

hexadecan-1-ol (hexadecyl alcohol, cetyl alcohol, palmityl alcohol) ethoxylated with 91 EO,

hexadecan-1-ol (hexadecyl alcohol, cetyl alcohol, palmityl alcohol) ethoxylated with 92 EO,

hexadecan-1-ol (hexadecyl alcohol, cetyl alcohol, palmityl alcohol) ethoxylated with 93 EO,

hexadecan-1-ol (hexadecyl alcohol, cetyl alcohol, palmityl alcohol) ethoxylated with 94 EO,

hexadecan-1-ol (hexadecyl alcohol, cetyl alcohol, palmityl alcohol) ethoxylated with 95 EO,

hexadecan-1-ol (hexadecyl alcohol, cetyl alcohol, palmityl alcohol) ethoxylated with 96 EO,

hexadecan-1-ol (hexadecyl alcohol, cetyl alcohol, palmityl alcohol) ethoxylated with 97 EO,

hexadecan-1-ol (hexadecyl alcohol, cetyl alcohol, palmityl alcohol) ethoxylated with 98 EO,

hexadecan-1-ol (hexadecyl alcohol, cetyl alcohol, palmityl alcohol) ethoxylated with 99 EO,

hexadecan-1-ol (hexadecyl alcohol, cetyl alcohol, palmityl alcohol) ethoxylated with 100 EO,

hexadecan-1-ol (hexadecyl alcohol, cetyl alcohol, palmityl alcohol) ethoxylated with 101 EO,

hexadecan-1-ol (hexadecyl alcohol, cetyl alcohol, palmityl alcohol) ethoxylated with 102 EO,

hexadecan-1-ol (hexadecyl alcohol, cetyl alcohol, palmityl alcohol) ethoxylated with 103 EO,

hexadecan-1-ol (hexadecyl alcohol, cetyl alcohol, palmityl alcohol) ethoxylated with 104 EO,

hexadecan-1-ol (hexadecyl alcohol, cetyl alcohol, palmityl alcohol) ethoxylated with 105 EO,

hexadecan-1-ol (hexadecyl alcohol, cetyl alcohol, palmityl alcohol) ethoxylated with 106 EO,

hexadecan-1-ol (hexadecyl alcohol, cetyl alcohol, palmityl alcohol) ethoxylated with 107 EO,

hexadecan-1-ol (hexadecyl alcohol, cetyl alcohol, palmityl alcohol) ethoxylated with 108 EO,

hexadecan-1-ol (hexadecyl alcohol, cetyl alcohol, palmityl alcohol) ethoxylated with 109 EO,

hexadecan-1-ol (hexadecyl alcohol, cetyl alcohol, palmityl alcohol) ethoxylated with 110 EO,

hexadecan-1-ol (hexadecyl alcohol, cetyl alcohol, palmityl alcohol) ethoxylated with 111 EO,

hexadecan-1-ol (hexadecyl alcohol, cetyl alcohol, palmityl alcohol) ethoxylated with 112 EO,

hexadecan-1-ol (hexadecyl alcohol, cetyl alcohol, palmityl alcohol) ethoxylated with 113 EO,

hexadecan-1-ol (hexadecyl alcohol, cetyl alcohol, palmityl alcohol) ethoxylated with 114 EO,

hexadecan-1-ol (hexadecyl alcohol, cetyl alcohol, palmityl alcohol) ethoxylated with 115 EO,

hexadecan-1-ol (hexadecyl alcohol, cetyl alcohol, palmityl alcohol) ethoxylated with 116 EO,

hexadecan-1-ol (hexadecyl alcohol, cetyl alcohol, palmityl alcohol) ethoxylated with 117 EO,

hexadecan-1-ol (hexadecyl alcohol, cetyl alcohol, palmityl alcohol) ethoxylated with 118 EO,

hexadecan-1-ol (hexadecyl alcohol, cetyl alcohol, palmityl alcohol) ethoxylated with 119 EO,

hexadecan-1-ol (hexadecyl alcohol, cetyl alcohol, palmityl alcohol) ethoxylated with 120 EO,

octadecan-1-ol (octadecyl alcohol, stearyl alcohol) ethoxylated with 50 EO,

octadecan-1-ol (octadecyl alcohol, stearyl alcohol) ethoxylated with 51 EO,

octadecan-1-ol (octadecyl alcohol, stearyl alcohol) ethoxylated with 52 EO,

octadecan-1-ol (octadecyl alcohol, stearyl alcohol) ethoxylated with 53 EO,

octadecan-1-ol (octadecyl alcohol, stearyl alcohol) ethoxylated with 54 EO,

octadecan-1-ol (octadecyl alcohol, stearyl alcohol) ethoxylated with 55 EO,

octadecan-1-ol (octadecyl alcohol, stearyl alcohol) ethoxylated with 56 EO,

octadecan-1-ol (octadecyl alcohol, stearyl alcohol) ethoxylated with 57 EO,

octadecan-1-ol (octadecyl alcohol, stearyl alcohol) ethoxylated with 58 EO,

octadecan-1-ol (octadecyl alcohol, stearyl alcohol) ethoxylated with 59 EO,

octadecan-1-ol (octadecyl alcohol, stearyl alcohol) ethoxylated with 60 EO,

octadecan-1-ol (octadecyl alcohol, stearyl alcohol) ethoxylated with 80 EO,

octadecan-1-ol (octadecyl alcohol, stearyl alcohol) ethoxylated with 81 EO,

octadecan-1-ol (octadecyl alcohol, stearyl alcohol) ethoxylated with 82 EO,

octadecan-1-ol (octadecyl alcohol, stearyl alcohol) ethoxylated with 83 EO,

octadecan-1-ol (octadecyl alcohol, stearyl alcohol) ethoxylated with 84 EO,

octadecan-1-ol (octadecyl alcohol, stearyl alcohol) ethoxylated with 85 EO,

octadecan-1-ol (octadecyl alcohol, stearyl alcohol) ethoxylated with 86 EO,

octadecan-1-ol (octadecyl alcohol, stearyl alcohol) ethoxylated with 87 EO,

octadecan-1-ol (octadecyl alcohol, stearyl alcohol) ethoxylated with 88 EO,

octadecan-1-ol (octadecyl alcohol, stearyl alcohol) ethoxylated with 89 EO,

octadecan-1-ol (octadecyl alcohol, stearyl alcohol) ethoxylated with 90 EO,

octadecan-1-ol (octadecyl alcohol, stearyl alcohol) ethoxylated with 91 EO,

octadecan-1-ol (octadecyl alcohol, stearyl alcohol) ethoxylated with 92 EO,

octadecan-1-ol (octadecyl alcohol, stearyl alcohol) ethoxylated with 93 EO,

octadecan-1-ol (octadecyl alcohol, stearyl alcohol) ethoxylated with 94 EO,

octadecan-1-ol (octadecyl alcohol, stearyl alcohol) ethoxylated with 95 EO,

octadecan-1-ol (octadecyl alcohol, stearyl alcohol) ethoxylated with 96 EO,

octadecan-1-ol (octadecyl alcohol, stearyl alcohol) ethoxylated with 97 EO,

octadecan-1-ol (octadecyl alcohol, stearyl alcohol) ethoxylated with 98 EO,

octadecan-1-ol (octadecyl alcohol, stearyl alcohol) ethoxylated with 99 EO,

octadecan-1-ol (octadecyl alcohol, stearyl alcohol) ethoxylated with 100 EO,

octadecan-1-ol (octadecyl alcohol, stearyl alcohol) ethoxylated with 101 EO,

octadecan-1-ol (octadecyl alcohol, stearyl alcohol) ethoxylated with 102 EO,

octadecan-1-ol (octadecyl alcohol, stearyl alcohol) ethoxylated with 103 EO,

octadecan-1-ol (octadecyl alcohol, stearyl alcohol) ethoxylated with 104 EO,

octadecan-1-ol (octadecyl alcohol, stearyl alcohol) ethoxylated with 105 EO,

octadecan-1-ol (octadecyl alcohol, stearyl alcohol) ethoxylated with 106 EO,

octadecan-1-ol (octadecyl alcohol, stearyl alcohol) ethoxylated with 107 EO,

octadecan-1-ol (octadecyl alcohol, stearyl alcohol) ethoxylated with 108 EO,

octadecan-1-ol (octadecyl alcohol, stearyl alcohol) ethoxylated with 109 EO,

octadecan-1-ol (octadecyl alcohol, stearyl alcohol) ethoxylated with 110 EO,

octadecan-1-ol (octadecyl alcohol, stearyl alcohol) ethoxylated with 111 EO,

octadecan-1-ol (octadecyl alcohol, stearyl alcohol) ethoxylated with 112 EO,

octadecan-1-ol (octadecyl alcohol, stearyl alcohol) ethoxylated with 113 EO,

octadecan-1-ol (octadecyl alcohol, stearyl alcohol) ethoxylated with 114 EO,

octadecan-1-ol (octadecyl alcohol, stearyl alcohol) ethoxylated with 115 EO,

octadecan-1-ol (octadecyl alcohol, stearyl alcohol) ethoxylated with 116 EO,

octadecan-1-ol (octadecyl alcohol, stearyl alcohol) ethoxylated with 117 EO,

octadecan-1-ol (octadecyl alcohol, stearyl alcohol) ethoxylated with 118 EO,

octadecan-1-ol (octadecyl alcohol, stearyl alcohol) ethoxylated with 119 EO,

octadecan-1-ol (octadecyl alcohol, stearyl alcohol) ethoxylated with 120 EO,

(9Z)-octadec-9-en-1-ol (oleyl alcohol) ethoxylated with 50 EO,

(9Z)-octadec-9-en-1-ol (oleyl alcohol) ethoxylated with 51 EO,

(9Z)-octadec-9-en-1-ol (oleyl alcohol) ethoxylated with 52 EO,

(9Z)-octadec-9-en-1-ol (oleyl alcohol) ethoxylated with 53 EO,

(9Z)-octadec-9-en-1-ol (oleyl alcohol) ethoxylated with 54 EO,

(9Z)-octadec-9-en-1-ol (oleyl alcohol) ethoxylated with 55 EO,

(9Z)-octadec-9-en-1-ol (oleyl alcohol) ethoxylated with 56 EO,

(9Z)-octadec-9-en-1-ol (oleyl alcohol) ethoxylated with 57 EO,

(9Z)-octadec-9-en-1-ol (oleyl alcohol) ethoxylated with 58 EO,

(9Z)-octadec-9-en-1-ol (oleyl alcohol) ethoxylated with 59 EO,

(9Z)-octadec-9-en-1-ol (oleyl alcohol) ethoxylated with 60 EO,

(9Z)-octadec-9-en-1-ol (oleyl alcohol) ethoxylated with 80 EO,

(9Z)-octadec-9-en-1-ol (oleyl alcohol) ethoxylated with 81 EO,

(9Z)-octadec-9-en-1-ol (oleyl alcohol) ethoxylated with 82 EO,

(9Z)-octadec-9-en-1-ol (oleyl alcohol) ethoxylated with 83 EO,

(9Z)-octadec-9-en-1-ol (oleyl alcohol) ethoxylated with 84 EO,

(9Z)-octadec-9-en-1-ol (oleyl alcohol) ethoxylated with 85 EO,

(9Z)-octadec-9-en-1-ol (oleyl alcohol) ethoxylated with 86 EO,

(9Z)-octadec-9-en-1-ol (oleyl alcohol) ethoxylated with 87 EO,

(9Z)-octadec-9-en-1-ol (oleyl alcohol) ethoxylated with 88 EO,

(9Z)-octadec-9-en-1-ol (oleyl alcohol) ethoxylated with 89 EO,

(9Z)-octadec-9-en-1-ol (oleyl alcohol) ethoxylated with 90 EO,

(9Z)-octadec-9-en-1-ol (oleyl alcohol) ethoxylated with 91 EO,

(9Z)-octadec-9-en-1-ol (oleyl alcohol) ethoxylated with 92 EO,

(9Z)-octadec-9-en-1-ol (oleyl alcohol) ethoxylated with 93 EO,

(9Z)-octadec-9-en-1-ol (oleyl alcohol) ethoxylated with 94 EO,

(9Z)-octadec-9-en-1-ol (oleyl alcohol) ethoxylated with 95 EO,

(9Z)-octadec-9-en-1-ol (oleyl alcohol) ethoxylated with 96 EO,

(9Z)-octadec-9-en-1-ol (oleyl alcohol) ethoxylated with 97 EO,

(9Z)-octadec-9-en-1-ol (oleyl alcohol) ethoxylated with 98 EO,

(9Z)-octadec-9-en-1-ol (oleyl alcohol) ethoxylated with 99 EO,

(9Z)-octadec-9-en-1-ol (oleyl alcohol) ethoxylated with 100 EO,

(9Z)-octadec-9-en-1-ol (oleyl alcohol) ethoxylated with 101 EO,

(9Z)-octadec-9-en-1-ol (oleyl alcohol) ethoxylated with 102 EO,

(9Z)-octadec-9-en-1-ol (oleyl alcohol) ethoxylated with 103 EO,

(9Z)-octadec-9-en-1-ol (oleyl alcohol) ethoxylated with 104 EO,

(9Z)-octadec-9-en-1-ol (oleyl alcohol) ethoxylated with 105 EO,

(9Z)-octadec-9-en-1-ol (oleyl alcohol) ethoxylated with 106 EO,

(9Z)-octadec-9-en-1-ol (oleyl alcohol) ethoxylated with 107 EO,

(9Z)-octadec-9-en-1-ol (oleyl alcohol) ethoxylated with 108 EO,

(9Z)-octadec-9-en-1-ol (oleyl alcohol) ethoxylated with 109 EO,

(9Z)-octadec-9-en-1-ol (oleyl alcohol) ethoxylated with 110 EO,

(9Z)-octadec-9-en-1-ol (oleyl alcohol) ethoxylated with 111 EO,

(9Z)-octadec-9-en-1-ol (oleyl alcohol) ethoxylated with 112 EO,

(9Z)-octadec-9-en-1-ol (oleyl alcohol) ethoxylated with 113 EO,

(9Z)-octadec-9-en-1-ol (oleyl alcohol) ethoxylated with 114 EO,

(9Z)-octadec-9-en-1-ol (oleyl alcohol) ethoxylated with 115 EO,

(9Z)-octadec-9-en-1-ol (oleyl alcohol) ethoxylated with 116 EO,

(9Z)-octadec-9-en-1-ol (oleyl alcohol) ethoxylated with 117 EO,

(9Z)-octadec-9-en-1-ol (oleyl alcohol) ethoxylated with 118 EO,

(9Z)-octadec-9-en-1-ol (oleyl alcohol) ethoxylated with 119 EO,

(9Z)-octadec-9-en-1-ol (oleyl alcohol) ethoxylated with 120 EO,

(9E)-octadec-9-en-1-ol (elaidyl alcohol) ethoxylated with 50 EO,

(9E)-octadec-9-en-1-ol (elaidyl alcohol) ethoxylated with 51 EO,

(9E)-octadec-9-en-1-ol (elaidyl alcohol) ethoxylated with 52 EO,

(9E)-octadec-9-en-1-ol (elaidyl alcohol) ethoxylated with 53 EO,

(9E)-octadec-9-en-1-ol (elaidyl alcohol) ethoxylated with 54 EO,

(9E)-octadec-9-en-1-ol (elaidyl alcohol) ethoxylated with 55 EO,

(9E)-octadec-9-en-1-ol (elaidyl alcohol) ethoxylated with 56 EO,

(9E)-octadec-9-en-1-ol (elaidyl alcohol) ethoxylated with 58 EO,

(9E)-octadec-9-en-1-ol (elaidyl alcohol) ethoxylated with 55 EO,

(9E)-octadec-9-en-1-ol (elaidyl alcohol) ethoxylated with 59 EO,

(9E)-octadec-9-en-1-ol (elaidyl alcohol) ethoxylated with 60 EO,

(9E)-octadec-9-en-1-ol (elaidyl alcohol) ethoxylated with 80 EO,

(9E)-octadec-9-en-1-ol (elaidyl alcohol) ethoxylated with 81 EO,

(9E)-octadec-9-en-1-ol (elaidyl alcohol) ethoxylated with 82 EO,

(9E)-octadec-9-en-1-ol (elaidyl alcohol) ethoxylated with 83 EO,

(9E)-octadec-9-en-1-ol (elaidyl alcohol) ethoxylated with 84 EO,

(9E)-octadec-9-en-1-ol (elaidyl alcohol) ethoxylated with 85 EO,

(9E)-octadec-9-en-1-ol (elaidyl alcohol) ethoxylated with 86 EO,

(9E)-octadec-9-en-1-ol (elaidyl alcohol) ethoxylated with 87 EO,

(9E)-octadec-9-en-1-ol (elaidyl alcohol) ethoxylated with 88 EO,

(9E)-octadec-9-en-1-ol (elaidyl alcohol) ethoxylated with 89 EO,

(9E)-octadec-9-en-1-ol (elaidyl alcohol) ethoxylated with 90 EO,

(9E)-octadec-9-en-1-ol (elaidyl alcohol) ethoxylated with 91 EO,

(9E)-octadec-9-en-1-ol (elaidyl alcohol) ethoxylated with 92 EO,

(9E)-octadec-9-en-1-ol (elaidyl alcohol) ethoxylated with 93 EO,

(9E)-octadec-9-en-1-ol (elaidyl alcohol) ethoxylated with 94 EO,

(9E)-octadec-9-en-1-ol (elaidyl alcohol) ethoxylated with 95 EO,

(9E)-octadec-9-en-1-ol (elaidyl alcohol) ethoxylated with 96 EO,

(9E)-octadec-9-en-1-ol (elaidyl alcohol) ethoxylated with 97 EO,

(9E)-octadec-9-en-1-ol (elaidyl alcohol) ethoxylated with 98 EO,

(9E)-octadec-9-en-1-ol (elaidyl alcohol) ethoxylated with 99 EO,

(9E)-octadec-9-en-1-ol (elaidyl alcohol) ethoxylated with 100 EO,

(9E)-octadec-9-en-1-ol (elaidyl alcohol) ethoxylated with 101 EO,

(9E)-octadec-9-en-1-ol (elaidyl alcohol) ethoxylated with 102 EO,

(9E)-octadec-9-en-1-ol (elaidyl alcohol) ethoxylated with 103 EO,

(9E)-octadec-9-en-1-ol (elaidyl alcohol) ethoxylated with 104 EO,

(9E)-octadec-9-en-1-ol (elaidyl alcohol) ethoxylated with 105 EO,

(9E)-octadec-9-en-1-ol (elaidyl alcohol) ethoxylated with 106 EO,

(9E)-octadec-9-en-1-ol (elaidyl alcohol) ethoxylated with 107 EO,

(9E)-octadec-9-en-1-ol (elaidyl alcohol) ethoxylated with 108 EO,

(9E)-octadec-9-en-1-ol (elaidyl alcohol) ethoxylated with 109 EO,

(9E)-octadec-9-en-1-ol (elaidyl alcohol) ethoxylated with 110 EO,

(9E)-octadec-9-en-1-ol (elaidyl alcohol) ethoxylated with 111 EO,

(9E)-octadec-9-en-1-ol (elaidyl alcohol) ethoxylated with 112 EO,

(9E)-octadec-9-en-1-ol (elaidyl alcohol) ethoxylated with 113 EO,

(9E)-octadec-9-en-1-ol (elaidyl alcohol) ethoxylated with 114 EO,

(9E)-octadec-9-en-1-ol (elaidyl alcohol) ethoxylated with 115 EO,

(9E)-octadec-9-en-1-ol (elaidyl alcohol) ethoxylated with 116 EO,

(9E)-octadec-9-en-1-ol (elaidyl alcohol) ethoxylated with 117 EO,

(9E)-octadec-9-en-1-ol (elaidyl alcohol) ethoxylated with 118 EO,

(9E)-octadec-9-en-1-ol (elaidyl alcohol) ethoxylated with 119 EO,

(9E)-octadec-9-en-1-ol (elaidyl alcohol) ethoxylated with 120 EO,

(9Z,12Z)-octadeca-9,12-dien-1-ol (linoleyl alcohol) ethoxylated with 50 EO,

(9Z,12Z)-octadeca-9,12-dien-1-ol (linoleyl alcohol) ethoxylated with 51 EO,

(9Z,12Z)-octadeca-9,12-dien-1-ol (linoleyl alcohol) ethoxylated with 52 EO,

(9Z,12Z)-octadeca-9,12-dien-1-ol (linoleyl alcohol) ethoxylated with 53 EO,

(9Z,12Z)-octadeca-9,12-dien-1-ol (linoleyl alcohol) ethoxylated with 54 EO,

(9Z,12Z)-octadeca-9,12-dien-1-ol (linoleyl alcohol) ethoxylated with 55 EO,

(9Z,12Z)-octadeca-9,12-dien-1-ol (linoleyl alcohol) ethoxylated with 56 EO,

(9Z,12Z)-octadeca-9,12-dien-1-ol (linoleyl alcohol) ethoxylated with 57 EO,

(9Z,12Z)-octadeca-9,12-dien-1-ol (linoleyl alcohol) ethoxylated with 58 EO,

(9Z,12Z)-octadeca-9,12-dien-1-ol (linoleyl alcohol) ethoxylated with 59 EO,

(9Z,12Z)-octadeca-9,12-dien-1-ol (linoleyl alcohol) ethoxylated with 60 EO,

(9Z,12Z)-octadeca-9,12-dien-1-ol (linoleyl alcohol) ethoxylated with 80 EO,

(9Z,12Z)-octadeca-9,12-dien-1-ol (linoleyl alcohol) ethoxylated with 81 EO,

(9Z,12Z)-octadeca-9,12-dien-1-ol (linoleyl alcohol) ethoxylated with 82 EO,

(9Z,12Z)-octadeca-9,12-dien-1-ol (linoleyl alcohol) ethoxylated with 83 EO,

(9Z,12Z)-octadeca-9,12-dien-1-ol (linoleyl alcohol) ethoxylated with 84 EO,

(9Z,12Z)-octadeca-9,12-dien-1-ol (linoleyl alcohol) ethoxylated with 85 EO,

(9Z,12Z)-octadeca-9,12-dien-1-ol (linoleyl alcohol) ethoxylated with 86 EO,

(9Z,12Z)-octadeca-9,12-dien-1-ol (linoleyl alcohol) ethoxylated with 87 EO,

(9Z,12Z)-octadeca-9,12-dien-1-ol (linoleyl alcohol) ethoxylated with 88 EO,

(9Z,12Z)-octadeca-9,12-dien-1-ol (linoleyl alcohol) ethoxylated with 89 EO,

(9Z,12Z)-octadeca-9,12-dien-1-ol (linoleyl alcohol) ethoxylated with 90 EO,

(9Z,12Z)-octadeca-9,12-dien-1-ol (linoleyl alcohol) ethoxylated with 91 EO,

(9Z,12Z)-octadeca-9,12-dien-1-ol (linoleyl alcohol) ethoxylated with 92 EO,

(9Z,12Z)-octadeca-9,12-dien-1-ol (linoleyl alcohol) ethoxylated with 93 EO,

(9Z,12Z)-octadeca-9,12-dien-1-ol (linoleyl alcohol) ethoxylated with 94 EO,

(9Z,12Z)-octadeca-9,12-dien-1-ol (linoleyl alcohol) ethoxylated with 95 EO,

(9Z,12Z)-octadeca-9,12-dien-1-ol (linoleyl alcohol) ethoxylated with 96 EO,

(9Z,12Z)-octadeca-9,12-dien-1-ol (linoleyl alcohol) ethoxylated with 97 EO,

(9Z,12Z)-octadeca-9,12-dien-1-ol (linoleyl alcohol) ethoxylated with 98 EO,

(9Z,12Z)-octadeca-9,12-dien-1-ol (linoleyl alcohol) ethoxylated with 99 EO,

(9Z,12Z)-octadeca-9,12-dien-1-ol (linoleyl alcohol) ethoxylated with 100 EO,

(9Z,12Z)-octadeca-9,12-dien-1-ol (linoleyl alcohol) ethoxylated with 101 EO,

(9Z,12Z)-octadeca-9,12-dien-1-ol (linoleyl alcohol) ethoxylated with 102 EO,

(9Z,12Z)-octadeca-9,12-dien-1-ol (linoleyl alcohol) ethoxylated with 103 EO,

(9Z,12Z)-octadeca-9,12-dien-1-ol (linoleyl alcohol) ethoxylated with 104 EO,

(9Z,12Z)-octadeca-9,12-dien-1-ol (linoleyl alcohol) ethoxylated with 105 EO,

(9Z,12Z)-octadeca-9,12-dien-1-ol (linoleyl alcohol) ethoxylated with 106 EO,

(9Z,12Z)-octadeca-9,12-dien-1-ol (linoleyl alcohol) ethoxylated with 107 EO,

(9Z,12Z)-octadeca-9,12-dien-1-ol (linoleyl alcohol) ethoxylated with 108 EO,

(9Z,12Z)-octadeca-9,12-dien-1-ol (linoleyl alcohol) ethoxylated with 109 EO,

(9Z,12Z)-octadeca-9,12-dien-1-ol (linoleyl alcohol) ethoxylated with 110 EO,

(9Z,12Z)-octadeca-9,12-dien-1-ol (linoleyl alcohol) ethoxylated with 111 EO,

(9Z,12Z)-octadeca-9,12-dien-1-ol (linoleyl alcohol) ethoxylated with 112 EO,

(9Z,12Z)-octadeca-9,12-dien-1-ol (linoleyl alcohol) ethoxylated with 113 EO,

(9Z,12Z)-octadeca-9,12-dien-1-ol (linoleyl alcohol) ethoxylated with 114 EO,

(9Z,12Z)-octadeca-9,12-dien-1-ol (linoleyl alcohol) ethoxylated with 115 EO,

(9Z,12Z)-octadeca-9,12-dien-1-ol (linoleyl alcohol) ethoxylated with 116 EO,

(9Z,12Z)-octadeca-9,12-dien-1-ol (linoleyl alcohol) ethoxylated with 117 EO,

(9Z,12Z)-octadeca-9,12-dien-1-ol (linoleyl alcohol) ethoxylated with 118 EO,

(9Z,12Z)-octadeca-9,12-dien-1-ol (linoleyl alcohol) ethoxylated with 119 EO,

(9Z,12Z)-octadeca-9,12-dien-1-ol (linoleyl alcohol) ethoxylated with 120 EO,

eicosan-1-ol (eicosyl alcohol, arachyl alcohol) ethoxylated with 50 EO,

eicosan-1-ol (eicosyl alcohol, arachyl alcohol) ethoxylated with 51 EO,

eicosan-1-ol (eicosyl alcohol, arachyl alcohol) ethoxylated with 52 EO,

eicosan-1-ol (eicosyl alcohol, arachyl alcohol) ethoxylated with 53 EO,

eicosan-1-ol (eicosyl alcohol, arachyl alcohol) ethoxylated with 54 EO,

eicosan-1-ol (eicosyl alcohol, arachyl alcohol) ethoxylated with 55 EO,

eicosan-1-ol (eicosyl alcohol, arachyl alcohol) ethoxylated with 56 EO,

eicosan-1-ol (eicosyl alcohol, arachyl alcohol) ethoxylated with 57 EO,

eicosan-1-ol (eicosyl alcohol, arachyl alcohol) ethoxylated with 58 EO,

eicosan-1-ol (eicosyl alcohol, arachyl alcohol) ethoxylated with 59 EO,

eicosan-1-ol (eicosyl alcohol, arachyl alcohol) ethoxylated with 60 EO,

eicosan-1-ol (eicosyl alcohol, arachyl alcohol) ethoxylated with 80 EO,

eicosan-1-ol (eicosyl alcohol, arachyl alcohol) ethoxylated with 81 EO,

eicosan-1-ol (eicosyl alcohol, arachyl alcohol) ethoxylated with 82 EO,

eicosan-1-ol (eicosyl alcohol, arachyl alcohol) ethoxylated with 83 EO,

eicosan-1-ol (eicosyl alcohol, arachyl alcohol) ethoxylated with 84 EO,

eicosan-1-ol (eicosyl alcohol, arachyl alcohol) ethoxylated with 85 EO,

eicosan-1-ol (eicosyl alcohol, arachyl alcohol) ethoxylated with 86 EO,

eicosan-1-ol (eicosyl alcohol, arachyl alcohol) ethoxylated with 87 EO,

eicosan-1-ol (eicosyl alcohol, arachyl alcohol) ethoxylated with 88 EO,

eicosan-1-ol (eicosyl alcohol, arachyl alcohol) ethoxylated with 89 EO,

eicosan-1-ol (eicosyl alcohol, arachyl alcohol) ethoxylated with 90 EO,

eicosan-1-ol (eicosyl alcohol, arachyl alcohol) ethoxylated with 91 EO,

eicosan-1-ol (eicosyl alcohol, arachyl alcohol) ethoxylated with 92 EO,

eicosan-1-ol (eicosyl alcohol, arachyl alcohol) ethoxylated with 93 EO,

eicosan-1-ol (eicosyl alcohol, arachyl alcohol) ethoxylated with 94 EO,

eicosan-1-ol (eicosyl alcohol, arachyl alcohol) ethoxylated with 95 EO,

eicosan-1-ol (eicosyl alcohol, arachyl alcohol) ethoxylated with 96 EO,

eicosan-1-ol (eicosyl alcohol, arachyl alcohol) ethoxylated with 97 EO,

eicosan-1-ol (eicosyl alcohol, arachyl alcohol) ethoxylated with 98 EO,

eicosan-1-ol (eicosyl alcohol, arachyl alcohol) ethoxylated with 99 EO,

eicosan-1-ol (eicosyl alcohol, arachyl alcohol) ethoxylated with 100 EO,

eicosan-1-ol (eicosyl alcohol, arachyl alcohol) ethoxylated with 101 EO,

eicosan-1-ol (eicosyl alcohol, arachyl alcohol) ethoxylated with 102 EO,

eicosan-1-ol (eicosyl alcohol, arachyl alcohol) ethoxylated with 103 EO,

eicosan-1-ol (eicosyl alcohol, arachyl alcohol) ethoxylated with 104 EO,

eicosan-1-ol (eicosyl alcohol, arachyl alcohol) ethoxylated with 105 EO,

eicosan-1-ol (eicosyl alcohol, arachyl alcohol) ethoxylated with 106 EO,

eicosan-1-ol (eicosyl alcohol, arachyl alcohol) ethoxylated with 107 EO,

eicosan-1-ol (eicosyl alcohol, arachyl alcohol) ethoxylated with 108 EO,

eicosan-1-ol (eicosyl alcohol, arachyl alcohol) ethoxylated with 109 EO,

eicosan-1-ol (eicosyl alcohol, arachyl alcohol) ethoxylated with 110 EO,

eicosan-1-ol (eicosyl alcohol, arachyl alcohol) ethoxylated with 111 EO,

eicosan-1-ol (eicosyl alcohol, arachyl alcohol) ethoxylated with 112 EO,

eicosan-1-ol (eicosyl alcohol, arachyl alcohol) ethoxylated with 113 EO,

eicosan-1-ol (eicosyl alcohol, arachyl alcohol) ethoxylated with 114 EO,

eicosan-1-ol (eicosyl alcohol, arachyl alcohol) ethoxylated with 115 EO,

eicosan-1-ol (eicosyl alcohol, arachyl alcohol) ethoxylated with 116 EO,

eicosan-1-ol (eicosyl alcohol, arachyl alcohol) ethoxylated with 117 EO,

eicosan-1-ol (eicosyl alcohol, arachyl alcohol) ethoxylated with 118 EO,

eicosan-1-ol (eicosyl alcohol, arachyl alcohol) ethoxylated with 119 EO,

eicosan-1-ol (eicosyl alcohol, arachyl alcohol) ethoxylated with 120 EO,

docosan-1-ol (docosyl alcohol, behenyl alcohol) ethoxylated with 50 EO,

docosan-1-ol (docosyl alcohol, behenyl alcohol) ethoxylated with 51 EO,

docosan-1-ol (docosyl alcohol, behenyl alcohol) ethoxylated with 52 EO,

docosan-1-ol (docosyl alcohol, behenyl alcohol) ethoxylated with 53 EO,

docosan-1-ol (docosyl alcohol, behenyl alcohol) ethoxylated with 54 EO,

docosan-1-ol (docosyl alcohol, behenyl alcohol) ethoxylated with 55 EO,

docosan-1-ol (docosyl alcohol, behenyl alcohol) ethoxylated with 56 EO,

docosan-1-ol (docosyl alcohol, behenyl alcohol) ethoxylated with 57 EO,

docosan-1-ol (docosyl alcohol, behenyl alcohol) ethoxylated with 58 EO,

docosan-1-ol (docosyl alcohol, behenyl alcohol) ethoxylated with 59 EO,

docosan-1-ol (docosyl alcohol, behenyl alcohol) ethoxylated with 60 EO

docosan-1-ol (docosyl alcohol, behenyl alcohol) ethoxylated with 80 EO,

docosan-1-ol (docosyl alcohol, behenyl alcohol) ethoxylated with 81 EO,

docosan-1-ol (docosyl alcohol, behenyl alcohol) ethoxylated with 82 EO,

docosan-1-ol (docosyl alcohol, behenyl alcohol) ethoxylated with 83 EO,

docosan-1-ol (docosyl alcohol, behenyl alcohol) ethoxylated with 84 EO,

docosan-1-ol (docosyl alcohol, behenyl alcohol) ethoxylated with 85 EO,

docosan-1-ol (docosyl alcohol, behenyl alcohol) ethoxylated with 86 EO,

docosan-1-ol (docosyl alcohol, behenyl alcohol) ethoxylated with 87 EO,

docosan-1-ol (docosyl alcohol, behenyl alcohol) ethoxylated with 88 EO,

docosan-1-ol (docosyl alcohol, behenyl alcohol) ethoxylated with 89 EO,

docosan-1-ol (docosyl alcohol, behenyl alcohol) ethoxylated with 90 EO,

docosan-1-ol (docosyl alcohol, behenyl alcohol) ethoxylated with 91 EO,

docosan-1-ol (docosyl alcohol, behenyl alcohol) ethoxylated with 92 EO,

docosan-1-ol (docosyl alcohol, behenyl alcohol) ethoxylated with 93 EO,

docosan-1-ol (docosyl alcohol, behenyl alcohol) ethoxylated with 94 EO,

docosan-1-ol (docosyl alcohol, behenyl alcohol) ethoxylated with 95 EO,

docosan-1-ol (docosyl alcohol, behenyl alcohol) ethoxylated with 96 EO,

docosan-1-ol (docosyl alcohol, behenyl alcohol) ethoxylated with 97 EO,

docosan-1-ol (docosyl alcohol, behenyl alcohol) ethoxylated with 98 EO,

docosan-1-ol (docosyl alcohol, behenyl alcohol) ethoxylated with 99 EO,

docosan-1-ol (docosyl alcohol, behenyl alcohol) ethoxylated with 100 EO,

docosan-1-ol (docosyl alcohol, behenyl alcohol) ethoxylated with 101 EO,

docosan-1-ol (docosyl alcohol, behenyl alcohol) ethoxylated with 102 EO,

docosan-1-ol (docosyl alcohol, behenyl alcohol) ethoxylated with 103 EO,

docosan-1-ol (docosyl alcohol, behenyl alcohol) ethoxylated with 104 EO,

docosan-1-ol (docosyl alcohol, behenyl alcohol) ethoxylated with 105 EO,

docosan-1-ol (docosyl alcohol, behenyl alcohol) ethoxylated with 106 EO,

docosan-1-ol (docosyl alcohol, behenyl alcohol) ethoxylated with 107 EO,

docosan-1-ol (docosyl alcohol, behenyl alcohol) ethoxylated with 108 EO,

docosan-1-ol (docosyl alcohol, behenyl alcohol) ethoxylated with 109 EO,

docosan-1-ol (docosyl alcohol, behenyl alcohol) ethoxylated with 110 EO,

docosan-1-ol (docosyl alcohol, behenyl alcohol) ethoxylated with 111 EO,

docosan-1-ol (docosyl alcohol, behenyl alcohol) ethoxylated with 112 EO,

docosan-1-ol (docosyl alcohol, behenyl alcohol) ethoxylated with 113 EO,

docosan-1-ol (docosyl alcohol, behenyl alcohol) ethoxylated with 114 EO,

docosan-1-ol (docosyl alcohol, behenyl alcohol) ethoxylated with 115 EO,

docosan-1-ol (docosyl alcohol, behenyl alcohol) ethoxylated with 116 EO,

docosan-1-ol (docosyl alcohol, behenyl alcohol) ethoxylated with 117 EO,

docosan-1-ol (docosyl alcohol, behenyl alcohol) ethoxylated with 118 EO

docosan-1-ol (docosyl alcohol, behenyl alcohol) ethoxylated with 119 EO

docosan-1-ol (docosyl alcohol, behenyl alcohol) ethoxylated with 120 EO.

A particularly advantageous oxidative composition as contemplated herein for coloring and/or lightening keratinic fibers is exemplified in that it contains one or more compounds from the following group as the ethoxylated fatty alcohol(s) of formula (IV):

hexadecan-1-ol (hexadecyl alcohol, cetyl alcohol, palmityl alcohol) ethoxylated with 50 EO,

hexadecan-1-ol (hexadecyl alcohol, cetyl alcohol, palmityl alcohol) ethoxylated with 51 EO,

hexadecan-1-ol (hexadecyl alcohol, cetyl alcohol, palmityl alcohol) ethoxylated with 52 EO,

hexadecan-1-ol (hexadecyl alcohol, cetyl alcohol, palmityl alcohol) ethoxylated with 53 EO,

hexadecan-1-ol (hexadecyl alcohol, cetyl alcohol, palmityl alcohol) ethoxylated with 54 EO,

hexadecan-1-ol (hexadecyl alcohol, cetyl alcohol, palmityl alcohol) ethoxylated with 55 EO,

hexadecan-1-ol (hexadecyl alcohol, cetyl alcohol, palmityl alcohol) ethoxylated with 56 EO,

hexadecan-1-ol (hexadecyl alcohol, cetyl alcohol, palmityl alcohol) ethoxylated with 57 EO,

hexadecan-1-ol (hexadecyl alcohol, cetyl alcohol, palmityl alcohol) ethoxylated with 58 EO,

hexadecan-1-ol (hexadecyl alcohol, cetyl alcohol, palmityl alcohol) ethoxylated with 59 EO,

hexadecan-1-ol (hexadecyl alcohol, cetyl alcohol, palmityl alcohol) ethoxylated with 60 EO,

hexadecan-1-ol (hexadecyl alcohol, cetyl alcohol, palmityl alcohol) ethoxylated with 90 EO,

hexadecan-1-ol (hexadecyl alcohol, cetyl alcohol, palmityl alcohol) ethoxylated with 91 EO,

hexadecan-1-ol (hexadecyl alcohol, cetyl alcohol, palmityl alcohol) ethoxylated with 92 EO,

hexadecan-1-ol (hexadecyl alcohol, cetyl alcohol, palmityl alcohol) ethoxylated with 93 EO,

hexadecan-1-ol (hexadecyl alcohol, cetyl alcohol, palmityl alcohol) ethoxylated with 94 EO,

hexadecan-1-ol (hexadecyl alcohol, cetyl alcohol, palmityl alcohol) ethoxylated with 95 EO,

hexadecan-1-ol (hexadecyl alcohol, cetyl alcohol, palmityl alcohol) ethoxylated with 96 EO,

hexadecan-1-ol (hexadecyl alcohol, cetyl alcohol, palmityl alcohol) ethoxylated with 97 EO,

hexadecan-1-ol (hexadecyl alcohol, cetyl alcohol, palmityl alcohol) ethoxylated with 98 EO,

hexadecan-1-ol (hexadecyl alcohol, cetyl alcohol, palmityl alcohol) ethoxylated with 99 EO,

hexadecan-1-ol (hexadecyl alcohol, cetyl alcohol, palmityl alcohol) ethoxylated with 100 EO,

hexadecan-1-ol (hexadecyl alcohol, cetyl alcohol, palmityl alcohol) ethoxylated with 101 EO,

hexadecan-1-ol (hexadecyl alcohol, cetyl alcohol, palmityl alcohol) ethoxylated with 102 EO,

hexadecan-1-ol (hexadecyl alcohol, cetyl alcohol, palmityl alcohol) ethoxylated with 103 EO,

hexadecan-1-ol (hexadecyl alcohol, cetyl alcohol, palmityl alcohol) ethoxylated with 104 EO,

hexadecan-1-ol (hexadecyl alcohol, cetyl alcohol, palmityl alcohol) ethoxylated with 105 EO,

hexadecan-1-ol (hexadecyl alcohol, cetyl alcohol, palmityl alcohol) ethoxylated with 106 EO,

hexadecan-1-ol (hexadecyl alcohol, cetyl alcohol, palmityl alcohol) ethoxylated with 107 EO,

hexadecan-1-ol (hexadecyl alcohol, cetyl alcohol, palmityl alcohol) ethoxylated with 108 EO,

hexadecan-1-ol (hexadecyl alcohol, cetyl alcohol, palmityl alcohol) ethoxylated with 109 EO,

hexadecan-1-ol (hexadecyl alcohol, cetyl alcohol, palmityl alcohol) ethoxylated with 110 EO,

octadecan-1-ol (octadecyl alcohol, stearyl alcohol) ethoxylated with 50 EO,

octadecan-1-ol (octadecyl alcohol, stearyl alcohol) ethoxylated with 51 EO,

octadecan-1-ol (octadecyl alcohol, stearyl alcohol) ethoxylated with 52 EO,

octadecan-1-ol (octadecyl alcohol, stearyl alcohol) ethoxylated with 53 EO,

octadecan-1-ol (octadecyl alcohol, stearyl alcohol) ethoxylated with 54 EO,

octadecan-1-ol (octadecyl alcohol, stearyl alcohol) ethoxylated with 55 EO,

octadecan-1-ol (octadecyl alcohol, stearyl alcohol) ethoxylated with 56 EO,

octadecan-1-ol (octadecyl alcohol, stearyl alcohol) ethoxylated with 57 EO,

octadecan-1-ol (octadecyl alcohol, stearyl alcohol) ethoxylated with 58 EO,

octadecan-1-ol (octadecyl alcohol, stearyl alcohol) ethoxylated with 59 EO,

octadecan-1-ol (octadecyl alcohol, stearyl alcohol) ethoxylated with 60 EO,

octadecan-1-ol (octadecyl alcohol, stearyl alcohol) ethoxylated with 90 EO,

octadecan-1-ol (octadecyl alcohol, stearyl alcohol) ethoxylated with 91 EO,

octadecan-1-ol (octadecyl alcohol, stearyl alcohol) ethoxylated with 92 EO,

octadecan-1-ol (octadecyl alcohol, stearyl alcohol) ethoxylated with 93 EO,

octadecan-1-ol (octadecyl alcohol, stearyl alcohol) ethoxylated with 94 EO,

octadecan-1-ol (octadecyl alcohol, stearyl alcohol) ethoxylated with 95 EO,

octadecan-1-ol (octadecyl alcohol, stearyl alcohol) ethoxylated with 96 EO,

octadecan-1-ol (octadecyl alcohol, stearyl alcohol) ethoxylated with 97 EO,

octadecan-1-ol (octadecyl alcohol, stearyl alcohol) ethoxylated with 98 EO,

octadecan-1-ol (octadecyl alcohol, stearyl alcohol) ethoxylated with 99 EO,

octadecan-1-ol (octadecyl alcohol, stearyl alcohol) ethoxylated with 100 EO,

octadecan-1-ol (octadecyl alcohol, stearyl alcohol) ethoxylated with 101 EO,

octadecan-1-ol (octadecyl alcohol, stearyl alcohol) ethoxylated with 102 EO,

octadecan-1-ol (octadecyl alcohol, stearyl alcohol) ethoxylated with 103 EO,

octadecan-1-ol (octadecyl alcohol, stearyl alcohol) ethoxylated with 104 EO,

octadecan-1-ol (octadecyl alcohol, stearyl alcohol) ethoxylated with 105 EO,

octadecan-1-ol (octadecyl alcohol, stearyl alcohol) ethoxylated with 106 EO,

octadecan-1-ol (octadecyl alcohol, stearyl alcohol) ethoxylated with 107 EO,

octadecan-1-ol (octadecyl alcohol, stearyl alcohol) ethoxylated with 108 EO,

octadecan-1-ol (octadecyl alcohol, stearyl alcohol) ethoxylated with 109 EO,

octadecan-1-ol (octadecyl alcohol, stearyl alcohol) ethoxylated with 110 EO.

As the seventh essential formulation ingredient the oxidative compositions as contemplated herein contain at least one polyol selected from C₂-C₉ alkanols with 2 to 6 hydroxyl groups and polyethylene glycols with 3 to 20 ethylene oxide units as well as mixtures thereof, preferably in a total amount of 1 to 10% by weight, especially preferably from about 2 to about 8% by weight, extremely preferably from about 3 to about 6% by weight.

Preferred C₂ to C₉ alkanols with 2 to 6 hydroxyl groups and/or polyethylene glycols with 3 to 20 ethylene oxide units as contemplated herein are selected from 1,2-propylene glycol, glycerol, butylene glycols such a 1,2-butylene glycol, 1,3-butylene glycol and 1,4-butylene glycol, pentylene glycols such as 1,2-pentanediol and 1,5-pentanediol, hexanediols such as 1,6-hexanediol and 2-methyl-1,3-propanediol (INCI: hexylene glycol), hexanetriols such as 1,2,6-hexanetriol, 1,2-octanediol, 1,8-octanediol, dipropylene glycol, tripropylene glycol, diglycerol, triglycerol, erythritol, sorbitol as well as mixtures of the aforementioned substances. Suitable water-soluble polyethylene glycols are selected from PEG-3, PEG-4, PEG-6, PEG-7, PEG-8, PEG-9, PEG-10, PEG-12, PEG-14, PEG-16, PEG-18 and PEG-20 as well as mixtures thereof, but PEG-3 to PEG-8 are preferred.

As the eighth essential formulation ingredient, the oxidative compositions as contemplated herein contain at least one oil. Preferably at least one oil is present in a total amount of from about 0.1 to about 5% by weight, especially preferably from about 0.2 to about 3% by weight, extremely preferably from about 0.5 to about 2.5% by weight, each based on the weight of the oxidative composition.

Preferred oils as contemplated herein are selected from natural and synthetic hydrocarbons, especially preferably from mineral oils, paraffin oils, C₈-C₃₀ isoparaffins, in particular isoeicosane, polyisobutenes and polydecenes which are available for example under the brand name Emery® 3004, 3006, 3010 or under the brand name Ethylflo® from Albemarle or Nexbase® 2004G from Nestle, also selected from C₈-C₁₆ isoparaffins, in particular isodecane, isododecane, isotetradecane and isohexadecane as well as mixtures thereof and 1,3-di-(2-ethylhexyl)cyclohexane (available for example under the brand name Cetiol® S from BASF).

Additional oils that are preferred as contemplated herein are selected from the benzoic acid esters of linear or branched C₈₋₂₂ alkanols. Especially preferred are benzoic acid C₁₂-C₁₅ alkyl esters, available for example as the commercial product Finsolv® TN, benzoic acid isostearyl ester available for example as the commercial product Finsolv® SB, ethyl hexyl benzoate available for example as the commercial product Finsolv® EB and benzoic acid octyl dodecyl ester available for example as the commercial product Finsolv® BOD.

Additional preferred oils as contemplated herein are selected from fatty alcohols with 6 to 30 carbon atoms, which may be unsaturated or branched and saturated or branched and unsaturated. The branched alcohols are often referred to as Guerbet alcohols because they can be obtained by the Guerbet reaction. Preferred alcohols are 2-hexyldecanol (Eutanol® G 16), 2-octyldodecanol (Eutanol® G), 2-ethylhexyl alcohol and isostearyl alcohol.

Additional preferred oils are selected from mixtures of Guerbet alcohols and Guerbet alcohol esters for example the commercial product Cetiol® PGL (2-hexyldecanol and 2-hexyldecyllaurate).

Additional preferred cosmetic oils as contemplated herein are selected from the triglycerides (=triple esters of glycerol) of linear or branched, saturated or unsaturated optionally hydroxylated C₈₋₃₀ fatty acids. The use of natural oils, for example, amaranth seed oil, apricot kernel oil, argan oil, avocado oil, babassu oil, cottonseed oil, borage seed oil, camelina oil, thistle oil, peanut oil, pomegranate oil, grapefruit seed oil, hemp oil, hazel nut oil, elder berry seed oil, red currant seed oil, jojoba oil, linseed oil, macadamia nut oil, corn germ oil, almond oil, manila oil, evening primrose oil, olive oil, palm oil, palm kernel oil, Brazil nut oil, pecan nut oil, peach seed oil, rapeseed oil, castor oil, sea buckthorn berry oil, sea buckthorn kernel oil, sesame oil, soy oil, sunflower oil, grape seed oil, walnut oil, wild rose oil, wheat germ oil and the liquid fractions of coconut oil and the like. However, synthetic triglyceride oils are also preferred, in particular capric/caprylic triglycerides, for example, the commercial products Myritol® 318, Myritol® 331 (BASF) or Miglyol® 812 (Hüls) with unbranched fatty acid radicals as well as glyceryl triisostearol with branched fatty acid radicals.

Additional particularly preferred cosmetic oils as contemplated herein are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Additional especially preferred cosmetic oils as contemplated herein are selected from the esters of the linear or branched, saturated or unsaturated fatty alcohols with 2 to 30 carbon atoms with linear or branched, saturated or unsaturated fatty acids with 2 to 30 carbon atoms, which may be hydroxylated. These include 2-hexyldecylstearate (Eutanol® G 16 S), 2-hexyldecyllaurate, isodecyl neopentanoate, isononyl isononanoate, 2-ethylhexyl palmitate (Cegesoft® C 24) and 2-ethylhexyl stearate (Cetio® 868). Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyl octanoic acid 2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Additional preferred cosmetic oils as contemplated herein are selected from the addition products of 1 to 5 propylene oxide units onto monovalent or polyvalent C₈₋₂₂ alkanols, such as octanol, decanol, decanediol, lauryl alcohol, myristyl alcohol and stearyl alcohol, for example, PPG-2 myristyl ether and PPG-3 myristyl ether (Witconol® APM).

Additional preferred cosmetic oils as contemplated herein are selected from the addition products of at least 6 ethylene oxide and/or propylene oxide units onto monovalent or polyvalent C₃₋₂₂ alkanols, such as glycerol, butanol, butanediol, myristyl alcohol and stearyl alcohol which may be esterified if desired for example PPG-14 butyl ether (Ucon Fluid® AP), PPG-9 butyl ether (Breox® B25), PPG-10 butanediol (Macol® 57), PPG-15 stearyl ether (Arlamol® E) and glycereth 7-diisononanoate.

Additional preferred cosmetic oils as contemplated herein are selected from the C₈-C₂₂ fatty alcohol esters monovalent or polyvalent C₂-C₇ hydroxycarboxylic acids, in particular the esters of glycolic acid, lactic acid, malic acid, tartaric acid, citric acid and salicylic acid. Such esters based on linear C_{14/15} alkanols, for example, C₁₂-C₁₅ alkyl lactate and C_{12/13} alkanols branched in position 2 can be ordered from the company Nordmann, Rassmann GmbH & Co., Hamburg under the brand name Cosmacol®, in particular the commercial products Cosmacol® ESI, Cosmacol® EMI and Cosmacol® ETI.

Additional preferred cosmetic oils as contemplated herein are selected from the symmetrical, asymmetrical or cyclic esters of carbonic acid with C₃₋₂₂ alkanols, C₃₋₂₂ alkanediols or C₃₋₂₂ alkanetriols, e.g., dicaprylylcarbonate (Cetiol® CC) or the esters according to the teaching of DE 19756454 A1, in particular glycerol carbonate. Additional cosmetic oils that may be preferred as contemplated herein are selected from the esters of dimers of unsaturated C₁₂-C₂₂ fatty acids (dimer fatty acids) with monovalent linear branched or cyclic C₂-C₁₈ alkanols or with polyvalent linear or branched C₂-C₆ alkanols.

Additional cosmetic oils that are suitable as contemplated herein are selected from the silicone oils including, for example, dialkyl and alkylarylsiloxanes such as, for example, cyclopentasiloxane, cyclohexasiloxane, dimethylpolysiloxane and methyl phenyl polysiloxane but also hexamethyldisiloxane, octamethyltrisiloxane and decamethyltetrasiloxane. Volatile silicone oils which may be cyclic may be preferred for example octamethylcyclotetrasiloxane, decamethylcyclopentasiloxane and dodecamethylcyclohexasiloxane as well as mixtures thereof such as those contained in the commercial products DC 244, 245, 344 and 345 from Dow Corning Also suitable are volatile linear silicone oils, in particular hexamethyldisiloxane (L₂), octamethyltrisiloxane (L₃), decamethyltetrasiloxane (L₄) as well as any mixtures of two or three of L₂, L₃ and/or L₄, preferably mixtures such as those contained in the commercial products DC 2-1184, Dow Corning® 200 (0.65 cSt) and Dow Corning® 200 (1.5 cSt) from Dow Corning Preferred nonvolatile silicone oils are selected from higher molecular linear dimethylpolysiloxanes, which are available commercially under the designation Dow Corning® 190, Dow Corning® 200 fluid with kinematic viscosities (25° C.) in the range of from about 5-100 cSt, preferably from about 5-50 cSt or from about 5-10 cSt and dimethylpolysiloxane with a kinematic viscosity (25° C.) of approx. 350 cSt. It may be extremely preferred as contemplated herein to use mixtures of the aforementioned oils.

Preferred oxidative compositions as contemplated herein are exemplified in that the cosmetic oil is selected from natural and synthetic hydrocarbons, especially preferably paraffin oils, C₁₈-C₃₀ isoparaffins, in particular isoeicosane, polyisobutenes and polydecenes, C₈-C₁₆ isoparaffins as well as 1,3-di-(2-ethylhexyl)cyclohexane; the benzoic acid esters of linear or branched C₈₋₂₂ alkanols; fatty alcohols with 6 to 30 carbon atoms, which may be unsaturated or branched and saturated or branched and unsaturated; triglycerides of linear or branched, saturated or unsaturated optionally hydroxylated C₈₋₃₀ fatty acids, in particular natural oils; the dicarboxylic acid esters of linear or branched C₂-C₁₀ alkanols; the esters of the linear or branched, saturated or unsaturated fatty alcohols with 2-30 carbon atoms with linear or branched, saturated or unsaturated fatty acids with 2-30 carbon atoms, which may be hydroxylated; the addition products of 1 to 5 propylene oxide units onto monovalent or polyvalent C₈₋₂₂ alkanols; the addition products of at least 6 ethylene oxide units and/or propylene oxide units onto monovalent or polyvalent C₃₋₂₂ alkanols; the C₈-C₂₂ fatty alcohol esters of monovalent or polyvalent C₂-C₇ hydroxycarboxylic acids; the symmetrical, asymmetrical or cyclic esters of carbonic acid with C₃₋₂₂ alkanols, C₃₋₂₂ alkanediols or C₃₋₂₂ alkanetriols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols; silicone oils as well as mixtures of the aforementioned substances.

Especially preferred oxidative compositions as contemplated herein are exemplified in that they contain at least one lamellar phase. The ingredients of the oxidative compositions as contemplated herein preferably form at least one lamellar phase, especially an L β-phase, which results in the fact that the ammonia dissolved in the aqueous phase remains bound in the water of the lamellar phase, thereby reducing its evaporation, when mixed with an ammonia-containing alkalizing composition, preferably in mixing with the soap gel-based alkalizing composition used as contemplated herein.

The oxidative composition as contemplated herein preferably has a viscosity in the range of from about 800-3500 mPas, especially preferably from about 1500-3000 mPas, each measured at 20° C. in a model MV2 Haake viscometer at a speed of 8 revolutions per minute.

For stabilization of the oxidizing agent during storage, it is especially preferable for the oxidative composition as contemplated herein to have an acidic pH, in particular a pH in the range of from about 2.5 to about 5.5, preferably from about 3.0 to about 5.0. Preferred acidifying agents include edible acids such as citric acid, acetic acid, malic acid or tartaric acid as well as dilute mineral acids in particular phosphoric acid.

To stabilize the oxidizing agent in the oxidative composition as contemplated herein, it is preferable to use so-called complexing agents, i.e., chelating agents. Complexing agents are substances capable of complexing metal ions, i.e., forming complexes with them. Preferred complexing agents include so-called chelating agents, i.e., substances that form cyclic compounds with metal ions, in which a single ligand occupies more than one coordination site on a central atom. The number of bound ligands depends on the coordination number of the central ion. Conventional chelating agents that are preferred within the scope of the present disclosure include, for example, polyoxycarboxylic acids, polyamines, ethylenediamine tetraacetic acid (EDTA), nitrilotriacetic acid (NTA) and hydroxyethane diphosphonic acids and/or the alkali salts thereof. Chelating agents that are preferred as contemplated herein include phosphonates, preferably hydroxyalkane phosphonates and/or aminoalkane phosphonates and in particular 1-hydroxyethane-1,1-diphosphonate (HEDP) and/or its disodium or tetrasodium salt and/or ethylenediamine tetramethylene phosphonate (EDTMP) and/or its hexasodium salt and/or diethylenetriamine pentamethylene phosphonate (DTPMP) and/or its heptasodium or octasodium salt. Dipicolinic acid is also preferably used as a chelating agent as contemplated herein. Agents containing a combination of an EDTA salt and HEDP and dipicolinic acid are especially preferred as contemplated herein.

Another subject matter of the present patent application is a kit for oxidative color change of keratinic fibers containing two compositions (A) and (B) that are kept separately from one another, wherein

the composition (B) contains an oxidative composition as contemplated herein or one preferred oxidative composition as contemplated herein, as described above, i.e., an oxidative composition containing:

from about 50-96% by weight, preferably from about 70-93% by weight, especially preferably from about 80-90% by weight water,

0.5-20% by weight hydrogen peroxide,

at least one linear saturated 1-alkanol with 12-30 carbon atoms preferably in a total amount of from about 2-8% by weight, especially preferably from about 3 to about 6.5% by weight,

at least one mono- or diester of ethylene glycol with palmitic acid and/or stearic acid as well as mixtures thereof,

wherein the at least one ethylene glycol fatty acid ester is preferably present in a total amount of from about 0.01-1% by weight, especially preferably from about 0.1 to about 0.8% by weight,

at least one ethoxylated fatty alcohol of formula (III)

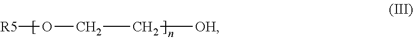

where R5 stands for a saturated or unsaturated, branched or unbranched C₈-C₂₄ alkyl group preferably for a saturated unbranched C₁₆ or C₁₈ alkyl group and
n stands for an integer from 10 to 30 preferably for an integer of 12 to 25, more preferably for an integer of 15 to 20,
wherein the at least one ethoxylated fatty alcohol of formula (III) is preferably present in a total amount of from about 0.25-2% by weight and especially preferably from about 0.5 to about 1.5% by weight,

at least one ethoxylated fatty alcohol of formula (IV)

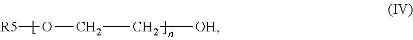

where R5 stands for a saturated or unsaturated, branched or unbranched C₈-C₂₄ alkyl group, preferably a saturated unbranched C₁₆ or C₁₈ alkyl group, and

n stands for an integer from 50 to 150 preferably for an integer from 80 to 120, more preferably for the number 100,

wherein the at least one ethoxylated fatty alcohol of formula (IV) is preferably present in a total amount of from about 0.03-0.3% by weight, especially preferably from about 0.08 to about 0.25% by weight, extremely preferably from about 0.1-0.2% by weight,

at least one polyol selected from C₂-C₉ alkanols with 2-6 hydroxyl groups and polyethylene glycols with 3-20 ethylene oxide units and mixtures thereof, preferably in a total amount of from about 1-10% by weight, especially preferably from about 2-8% by weight, extremely preferably from about 3-6% by weight and

at least one oil, preferably in a total amount of from about 0.1-5% by weight, especially preferably from about 0.2-3% by weight, extremely preferably from about 0.5-2.5% by weight,

wherein all the quantitative amounts are based on the weight of the oxidative composition;
and the composition (A) is in the form of a soap-based gel and contains

from about 25-85% by weight water,

ammonia,

from about 5-20% by weight, preferably from about 8-16% by weight of at least one salt of a C₁₂-C₂₂ fatty acid, preferably a salt of oleic acid,

from about 2-20% by weight, preferably from about 3-16% by weight of at least one polyethylene glycol ether of a linear saturated or unsaturated C₁₀-C₁₈ alkanol with 1-5 ethylene oxide units in the molecule,

from about 0-1% by weight of at least one oil,

preferably at least one organic solvent, selected from monovalent to tetravalent C₂-C₆ alcohols, especially preferably in a total amount of from about 0.1-35% by weight,

optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule,

and

optionally at least one oxidative dye precursor

and has a pH in the range of from about 8 to about 11.5 measured at 20° C.,
wherein all the quantitative amounts are based on the weight of the composition (A);
wherein the compositions (A) and (B) are preferably present in a weight-based ratio (A)/(B) in the range of from about 0.33-3, especially preferably from about 0.5-2, extremely preferably 1:1.

The composition (A) corresponds to the alkalizing preparation described above.

The composition (A) contains ammonia and optionally at least one additional alkalizing agent selected from the group formed by alkanolamines, basic amino acids as well as inorganic alkalizing agents such as alkaline (earth) metal hydroxides, alkaline (earth) metal metasilicates, alkaline (earth) metal phosphates and alkaline (earth) metal hydrogen phosphates. Suitable inorganic alkalizing agents included sodium hydroxide, potassium hydroxide, sodium silicate and sodium metasilicate. Organic alkalizing agents that can be used as contemplated herein are selected from monoethanolamine, 2-amino-2-methylpropanol and triethanolamine. The basic amino acids that can be used as the alkalizing agent as contemplated herein are preferably selected from the group formed from arginine, lysine, ornithine and histidine, especially preferably arginine. Ammonia (NH₃) is usually used in the form of its aqueous solution. Aqueous ammonia solutions often contain ammonia (NH₃) in concentrations of from about 10 to about 32% by weight. The use of an aqueous ammonia solution containing 25% by weight ammonia (NH₃) is preferred here. The total amount of alkalizing agent is preferably selected so that the mixture, i.e., the ready-to-use color changing agent, has an alkaline pH, preferably a pH of from about 8 to about 11.5, especially preferably a pH of from about 8.5 to about 11, extremely preferably a pH of from about 9.0 to about 10.5. Ammonia and optionally monoethanolamine are preferably present in the composition (A) used as contemplated herein in amounts of from about 0.01-10% by weight, preferably from about 0.1 to about 7.5% by weight, more preferably from about 0.2 to about 5.5% by weight and especially preferably from about 0.4 to about 4.5% by weight, each based on the weight of composition (A), so that the pH of the composition (A) is in the range of from about 8 to about 11.5, measured at 20° C.

The alkalizing composition (A) used as contemplated herein contains at least one oxidative dye precursor which is preferably selected from one or more developer components and optionally one or more coupler components as the optional ingredient.

Preferably at least one oxidative dye precursor is present in a total amount of from about 0.0001 to about 10.0% by weight, preferably from about 0.001 to about 8% by weight, each based on the weight of the composition (A).

It may be preferred as contemplated herein to select as the developer component at least one compound from the group formed by p-phenylenediamine, p-toluylenediamine, 2-(2-hydroxyethyl)-p-phenylenediamine, 2-(1,2-dihydroxyethyl)-p-phenylenediamine, N,N-bis-(2-hydroxyethyl)-p-phenylenediamine, N-(4-amino-3-methylphenyl)-N-[3-(1H-imidazol-1-yl)propyl]amine, N,N′-bis-(2-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(1,2-dihydroxyethyl)phenol, 4-amino-2-(diethylaminomethyl)phenol, 4,5-diamino-1-(2-hydroxyethyl)pyrazole, 2,4,5,6-tetraaminopyrimidine, 4-hydroxy-2,5,6-triaminopyrimidine, 2-hydroxy-4,5,6-triaminopyrimidine as well as their physiologically tolerable salts.

Preferably at least one developer component is present in a total amount of from about 0.0001 to about 10.0% by weight, preferably from about 0.001 to about 8% by weight, each based on the weight of the composition (A).

Coupler components within the scope of oxidative dyeing alone do not form a significant coloration on their own but instead always require the presence of developer components. It is therefore preferred as contemplated herein that when that when using at least one developer component, in addition at least one coupler component is also used.

Preferred coupler components for use as contemplated herein are selected from 3-aminophenol, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, 2,4-dichloro-3-aminophenol, 2-(2,4-diaminophenoxy)ethanol, 1,3-bis-(2,4-diaminophenoxy)propane, 1-methoxy-2-amino-4-(2-hydroxyethylamino)benzene, 1,3-bis-(2,4-diaminophenyl)propane, 2,6-bis-(2′-hydroxyethylamino)-1-methylbenzene, 2-({3-[(2-hydroxyethyl)amino]-4-methoxy-5-methylphenyl}amino)ethanol, 2-({3-[(2-hydroxyethyl)amino]-2-methoxy-5-methylphenyl}amino)ethanol, 2-({3-[(2-hydroxyethyl)amino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(2-hydroxyethyl)aminobenzene, resorcinol, resorcinol monomethyl ether, 2-methylresorcinol, 5-methylresorcinol, 2,5-dimethylresorcinol, 2-chlororesorcinol, 4-chlororesorcinol, pyrogallol, 1,2,4-trihydroxybenzene, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine, 3,5-diamino-2,6-dimethoxypyridine, 2,6-dihydroxy-3,4-dimethylpyridine, 3,4-diaminopyridine, 2-(2-methoxyethyl)amino-3-amino-6-methoxypyridine, 2-(4′-methoxyphenyl)amino-3-aminopyridine, 1-naphthol, 2-methyl-1-naphthol, 2-hydroxymethyl-1-naphthol, 2-hydroxyethyl-1-naphthol, 1,3-dihydroxynaphthalene, 1,5-dihydroxynaphthalene, 1,6-dihydroxynaphthalene, 1,7-dihydroxynaphthalene, 1,8-dihydroxynaphthalene, 2,7-dihydroxynaphthalene, 2,3-dihydroxynaphthalene, 4-hydroxyindole, 6-hydroxyindole, 7-hydroxyindole, 4-hydroxyindoline, 6-hydroxyindoline, 7-hydroxyindoline, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine and 4,6-dihydroxy-2-methylpyrimidine or mixtures of these compounds or their physiologically tolerable salts.

Preferably at least one coupler component is present in a total amount of from about 0.0001 to about 10.0% by weight, preferably from about 0.001 to about 8% by weight, each based on the weight of the composition (A).

Developer components and coupler components are generally used in approximately equimolar amounts relative to one another. If the equimolar use has proven expedient, then a certain excess of individual oxidative dye precursors is not a disadvantage, so that developer components and coupler components may be present in a molar ratio of from about 1:0.5 to about 1:3 in particular from about 1:1 to about 1:2.

Kits that are preferred as contemplated herein for oxidative color change of keratinic fibers are exemplified in that the aforementioned oxidative composition (B) as contemplated herein or one that is preferred as contemplated herein and the aforementioned alkalizing composition (A) are present in a weight ratio (A)/(B) of from about 0.33 to about 3, especially preferably of from about 0.5 to about 2 and extremely preferably in a weight ratio of 1:1.

Kits for oxidative color change of keratinic fibers that are especially preferred as contemplated herein are exemplified in that the aforementioned oxidative composition (B) as contemplated herein or one that is preferred as contemplated herein and the aforementioned alkalizing composition (A) are present in a weight ratio (A)/(B) of from about 0.33 to about 3, especially preferably from about 0.5 to about 2 and extremely preferably in a weight ratio of 1:1, wherein the kit does not contain any other components that are added to the ready-to-use color change mixture, while components for pretreatment or after-treatment of the keratinic fibers for example conditioner or shampoos may already be present in the kit.

In an embodiment that is preferred as contemplated herein, the kit as contemplated herein is also exemplified in that the composition (A) contains, each based on its weight, from about 25-85% by weight water, ammonia, from about 5-20% by weight, preferably from about 8-16% by weight of at least one salt of a C₁₂-C₂₂ fatty acid, preferably a salt of oleic acid, optionally from about 0.1-3% by weight of an anionic surfactant, selected from alkyl sulfates, alkyl ether sulfates and ether carboxylic acids with 10 to 20 carbon atoms in the alkyl group and up to 16 glycol ether groups in the molecule, from about 2-20% by weight, preferably from about 3-16% by weight of at least one polyethylene glycol ether of a linear saturated or unsaturated C₁₀-C₁₈ alkanol with 1 to 5 ethylene oxide units in the molecule, from about 0-1% by weight of at least one oil, preferably at least one organic solvent selected from monovalent to tetravalent C₂-C₆ alcohols, especially preferably in a total amount of from about 0.1-35% by weight and optionally at least one oxidative dye precursor and having a pH in the range of from about 8 to about 11.5, measured at 20° C.

With respect to additional preferred embodiments of the kit as contemplated herein, what was already said regarding the oxidative compositions as contemplated herein as well as those preferred as contemplated herein and the alkalizing compositions (A) that are used as contemplated herein also applies here, mutatis mutandis.

Another subject matter of the present patent application is a method for oxidative color change of keratinic fibers and is exemplified by the following methods steps:

Providing an oxidative composition (B) according to any one of claims 1 to 8, containing:

from about 50 to about 96% by weight, preferably from about 70 to about 93% by weight, especially preferably from about 80 to about 90% by weight water,

from about 0.5 to about 20% by weight hydrogen peroxide,

at least one linear saturated 1-alkanol with 12 to 30 carbon atoms preferably in a total amount of from about 2 to about 8% by weight, especially preferably from about 3 to about 6.5% by weight,

at least one mono- or diester of ethylene glycol with palmitic acid and/or stearic acid as well as mixtures thereof,

wherein the at least one ethylene glycol fatty acid ester is preferably present in a total amount of from about 0.01-1% by weight, especially preferably from about 0.1 to about 0.8% by weight,

at least one ethoxylated fatty alcohol of the formula (III):

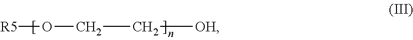

where R5 stands for a branched or unbranched, saturated or unsaturated C₈-C₂₄ alkyl group, preferably for a saturated, unbranched C₁₆ or C₁₈ alkyl group and n stands for an integer from 10 to 30, preferably for an integer from 12 to 25, more preferably for an integer from 15 to 20, wherein the at least one ethoxylated fatty alcohol of formula (III) is preferably present in a total amount of from about 0.25-2% by weight, especially preferably from about 0.5 to 1.5% by weight,

at least one ethoxylated fatty alcohol of formula (IV):

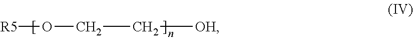

where R5 stands for a branched or unbranched, saturated or unsaturated C₈-C₂₄ alkyl group, preferably for a saturated unbranched C₁₆ or C₁₈ alkyl group and n stands for an integer from about 50 to about 150, preferably for an integer from about 80 to about 120, more preferably for the integer 100, wherein the at least one ethoxylated fatty alcohol of formula (IV) is preferably present in a total amount of from about 0.03-0.3% by weight, especially preferably from about 0.08 to about 0.25% by weight, extremely preferably from about 0.1-0.2% by weight,

at least one polyol selected from C₂-C₉ alkanols with 2-6 hydroxyl groups and polyethylene glycols with 3-20 ethylene oxide units and mixtures thereof, preferably in a total amount of from about 1-10% by weight, especially preferably from about 2-8% by weight, extremely preferably from about 3-6% by weight, and

at least one oil, preferably in a total amount of from about 0.1-5% by weight, especially preferably from about 0.2-3% by weight, extremely preferably from about 0.5-2.5% by weight,

wherein all the quantitative amounts are based on the weight of the oxidative composition and the composition (A) is in the form of a soap-based gel and contains:

from about 25-85% by weight water,

ammonia,

from about 5-20% by weight, preferably from about 8-16% by weight of at least one salt of a C₁₂-C₂₂ fatty acid, preferably a salt of oleic acid,

from about 2-20% by weight, preferably from about 3-16% by weight of at least one polyethylene glycol ether of a linear saturated or unsaturated C₁₀-C₁₈ alkanol with 1 to 5 ethylene oxide units in the molecule,

a total of from about 0-1% by weight of at least one oil,

at least one organic solvent, selected from monovalent to tetravalent C₂-C₆ alcohols, especially preferably in a total amount of from about 0.1-35% by weight,

optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule and

optionally at least one oxidative dye precursor

and has a pH in the range of from about 8 to about 11.5 measured at 20° C.,
wherein all the quantitative amounts are based on the weight of the composition (A);
preparation of a mixture of the aforementioned composition (B) and the aforementioned composition (A), preferably in a weight-based mixing ratio (A)/(B) in the range of from about 0.33-3, especially preferably from about 0.5-2, extremely preferably 1:1,
then immediately thereafter distribution of the ready-to-use agent on the fiber,
leaving the agent on the fibers for a period of from about 1 to about 60 minutes, then immediately thereafter rinsing the remaining agent out of the fibers and optionally drying the fibers.

Methods of oxidative color change of keratinic fibers that are preferred as contemplated herein are exemplified in that the aforementioned oxidative composition (B) and the aforementioned alkalizing composition (A) are mixed with one another in the weight-based mixing ratio (A)/(B) in the range of from about 0.33-3, especially preferably from about 0.5-2, extremely preferably 1:1.

With respect to additional preferred embodiments of the method as contemplated herein, what was said above regarding the oxidative compositions as contemplated herein as well as those preferred as contemplated herein and also what was said about the alkalizing compositions (A) that are used as contemplated herein is also applicable.

The terms keratinic fibers and/or keratin-containing fibers according to the present disclosure are understood to refer to wool, skins, feathers and human hair in particular. The coloring and/or lightening methods as contemplated herein may also be selected from other natural fibers in principle (for example, cotton, jute, sisal, linen, silk or modified natural fibers, such as, for example, regenerated cellulose, nitro, alkyl or hydroxyalkyl or acetyl cellulose.

The ready-to-use dye of the method as contemplated herein is preferably prepared by combining the oxidative composition as contemplated herein or one that is preferred as contemplated herein with an alkalizing composition (A) as contemplated herein in a reclosable container and then mixing them thoroughly.

In the next method step, the ready-to-use dye is distributed on the keratinic fibers. In the method for changing the color of human hair, the ready-to-use agent is distributed directly on the head hair of the user. The distribution is preferably carried out manually. To do so, the user removes the ready-to-use agent from the mixing container, preferably the resealable container, by scooping or pouring it directly into one's hand and then distributing and preferably incorporating the agent on and into the scalp hair. The local contact between the ready-to-use color change agent and the user's hands is preferably prevented by using suitable gloves such as disposable gloves, for example, gloves made of latex.

Then the ready-to-use dye remains on the fibers to be treated for a period of from about 1 to about 60 min. The period of time is especially preferably in the range of from about 10 to about 45 min, especially preferably from about 20 to about 30 min.

The application temperatures may be in a range between about 15 and about 40° C. If necessary, while the agent remains on the fibers, a higher temperature or a precisely defined temperature can also be adjusted by employing external heat sources. It is especially preferred to support the color change through physical measures. Methods as contemplated herein, in which application of the agent is supported by the action of heat, IR and UV radiation during the treatment time may therefore be preferred.

After the treatment time has elapsed, the ready-to-use dye and/or the remaining coloring agent is removed from the fibers to be treated by rinsing it out in the last process step. To do so the fibers are rinsed out with water and/or an aqueous surfactant preparation. This is usually done using hot water at from about 20° C. to about 40° C. and/or a suitably heated aqueous surfactant preparation. Optionally this may be followed by additional treatment steps such as application of a leave-on or rinse-off conditioner, another coloring step, e.g., dyeing or lightening strands, a hair shaping and/or hair drying step.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An oxidation composition for oxidative treatment of hair, wherein the oxidation composition comprises:
from about 50-96% by weight water,
from about 0.5-20% by weight hydrogen peroxide,
at least one linear saturated 1-alkanol with 12-30 carbon atoms
at least one mono- or diester of ethylene glycol with palmitic acid and/or stearic acid as well as mixtures thereof,
at least one ethoxylated fatty alcohol of the formula (III) 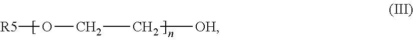
where R5 stands for a saturated or unsaturated, branched or unbranched C₈-C₂₄ alkyl group and n stands for an integer from 10 to 30,
at least one ethoxylated fatty alcohol of formula (IV) 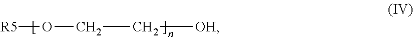
where R5 stands for a saturated or unsaturated, branched or unbranched C₈-C₂₄ alkyl group and n stands for an integer from about 50 to about 150,
at least one polyol selected from C₂-C₉ alkanols with 2-6 hydroxyl groups, polyethylene glycols with 3-20 ethylene oxide units, or mixtures thereof, and
at least one oil,
wherein all the quantitative amounts are based on the weight of the oxidative composition.

2. The composition according to claim 1, wherein the at least one linear saturated 1-alkanol with 12-30 carbon atoms is selected from lauryl alcohol, myristyl alcohol, cetyl alcohol, stearyl alcohol, arachidyl alcohol, behenyl alcohol, or mixtures of these alkanols.

3. The composition according to claim 1, wherein at least one compound selected from ethylene glycol monostearate, ethylene glycol distearate, ethylene glycol monopalmitate and ethylene glycol dipalmitate in a total amount of from about 0.01 to about 1% by weight is used as the ethylene glycol fatty acid ester.

4. The composition according to claim 1, wherein at least one compound selected from the following is used as the ethoxylated fatty alcohol of formula (III)
decan-1-ol (decyl alcohol) ethoxylated with 10 EO,
decan-1-ol (decyl alcohol) ethoxylated with 20 EO,
decan-1-ol (decyl alcohol) ethoxylated with 30 EO,
dodecan-1-ol (dodecyl alcohol, lauryl alcohol) ethoxylated with 10 EO,
dodecan-1-ol (dodecyl alcohol, lauryl alcohol) ethoxylated with 20 EO,
dodecan-1-ol (dodecyl alcohol, lauryl alcohol) ethoxylated with 30 EO,
hexadecan-1-ol (hexadecyl alcohol, cetyl alcohol, palmityl alcohol) ethoxylated with 10 EO,
hexadecan-1-ol (hexadecyl alcohol, cetyl alcohol, palmityl alcohol) ethoxylated with 12 EO,
hexadecan-1-ol (hexadecyl alcohol, cetyl alcohol, palmityl alcohol) ethoxylated with 15 EO,
hexadecan-1-ol (hexadecyl alcohol, cetyl alcohol, palmityl alcohol) ethoxylated with 20 EO,
hexadecan-1-ol (hexadecyl alcohol, cetyl alcohol, palmityl alcohol) ethoxylated with 25 EO,
hexadecan-1-ol (hexadecyl alcohol, cetyl alcohol, palmityl alcohol) ethoxylated with 30 EO,
octadecan-1-ol (octadecyl alcohol, stearyl alcohol) ethoxylated with 10 EO,
octadecan-1-ol (octadecyl alcohol, stearyl alcohol) ethoxylated with 12 EO,
octadecan-1-ol (octadecyl alcohol, stearyl alcohol) ethoxylated with 15 EO,
octadecan-1-ol (octadecyl alcohol, stearyl alcohol) ethoxylated with 20 EO,
octadecan-1-ol (octadecyl alcohol, stearyl alcohol) ethoxylated with 25 EO,
octadecan-1-ol (octadecyl alcohol, stearyl alcohol) ethoxylated with 30 EO,
eicosan-1-ol (eicosyl alcohol, arachyl alcohol) ethoxylated with 10 EO,
eicosan-1-ol (eicosyl alcohol, arachyl alcohol) ethoxylated with 12 EO,
eicosan-1-ol (eicosyl alcohol, arachyl alcohol) ethoxylated with 15 EO,
eicosan-1-ol (eicosyl alcohol, arachyl alcohol) ethoxylated with 20 EO,
eicosan-1-ol (eicosyl alcohol, arachyl alcohol) ethoxylated with 25 EO,
eicosan-1-ol (eicosyl alcohol, arachyl alcohol) ethoxylated with 30 EO,
docosan-1-ol (docosyl alcohol, behenyl alcohol) ethoxylated with 10 EO
docosan-1-ol (docosyl alcohol, behenyl alcohol) ethoxylated with 12 EO
docosan-1-ol (docosyl alcohol, behenyl alcohol) ethoxylated with 15 EO
docosan-1-ol (docosyl alcohol, behenyl alcohol) ethoxylated with 20 EO
docosan-1-ol (docosyl alcohol, behenyl alcohol) ethoxylated with 25 EO
docosan-1-ol (docosyl alcohol, behenyl alcohol) ethoxylated with 30 EO, as well as mixtures thereof.

5. The composition according to claim 1, wherein at least one compound selected from the following is used as the ethoxylated fatty alcohol of formula (IV)
hexadecan-1-ol (hexadecyl alcohol, cetyl alcohol, palmityl alcohol) ethoxylated with 50 EO,
hexadecan-1-ol (hexadecyl alcohol, cetyl alcohol, palmityl alcohol) ethoxylated with 51 EO,
hexadecan-1-ol (hexadecyl alcohol, cetyl alcohol, palmityl alcohol) ethoxylated with 52 EO,
hexadecan-1-ol (hexadecyl alcohol, cetyl alcohol, palmityl alcohol) ethoxylated with 53 EO,
hexadecan-1-ol (hexadecyl alcohol, cetyl alcohol, palmityl alcohol) ethoxylated with 54 EO,
hexadecan-1-ol (hexadecyl alcohol, cetyl alcohol, palmityl alcohol) ethoxylated with 55 EO,
hexadecan-1-ol (hexadecyl alcohol, cetyl alcohol, palmityl alcohol) ethoxylated with 56 EO,
hexadecan-1-ol (hexadecyl alcohol, cetyl alcohol, palmityl alcohol) ethoxylated with 57 EO,
hexadecan-1-ol (hexadecyl alcohol, cetyl alcohol, palmityl alcohol) ethoxylated with 58 EO,
hexadecan-1-ol (hexadecyl alcohol, cetyl alcohol, palmityl alcohol) ethoxylated with 59 EO,
hexadecan-1-ol (hexadecyl alcohol, cetyl alcohol, palmityl alcohol) ethoxylated with 60 EO,
hexadecan-1-ol (hexadecyl alcohol, cetyl alcohol, palmityl alcohol) ethoxylated with 90 EO,
hexadecan-1-ol (hexadecyl alcohol, cetyl alcohol, palmityl alcohol) ethoxylated with 91 EO,
hexadecan-1-ol (hexadecyl alcohol, cetyl alcohol, palmityl alcohol) ethoxylated with 92 EO,
hexadecan-1-ol (hexadecyl alcohol, cetyl alcohol, palmityl alcohol) ethoxylated with 93 EO,
hexadecan-1-ol (hexadecyl alcohol, cetyl alcohol, palmityl alcohol) ethoxylated with 94 EO,
hexadecan-1-ol (hexadecyl alcohol, cetyl alcohol, palmityl alcohol) ethoxylated with 95 EO,
hexadecan-1-ol (hexadecyl alcohol, cetyl alcohol, palmityl alcohol) ethoxylated with 96 EO,
hexadecan-1-ol (hexadecyl alcohol, cetyl alcohol, palmityl alcohol) ethoxylated with 97 EO,
hexadecan-1-ol (hexadecyl alcohol, cetyl alcohol, palmityl alcohol) ethoxylated with 98 EO,
hexadecan-1-ol (hexadecyl alcohol, cetyl alcohol, palmityl alcohol) ethoxylated with 99 EO,
hexadecan-1-ol (hexadecyl alcohol, cetyl alcohol, palmityl alcohol) ethoxylated with 100 EO,
hexadecan-1-ol (hexadecyl alcohol, cetyl alcohol, palmityl alcohol) ethoxylated with 101 EO,
hexadecan-1-ol (hexadecyl alcohol, cetyl alcohol, palmityl alcohol) ethoxylated with 102 EO,
hexadecan-1-ol (hexadecyl alcohol, cetyl alcohol, palmityl alcohol) ethoxylated with 103 EO,
hexadecan-1-ol (hexadecyl alcohol, cetyl alcohol, palmityl alcohol) ethoxylated with 104 EO,
hexadecan-1-ol (hexadecyl alcohol, cetyl alcohol, palmityl alcohol) ethoxylated with 105 EO,
hexadecan-1-ol (hexadecyl alcohol, cetyl alcohol, palmityl alcohol) ethoxylated with 106 EO,
hexadecan-1-ol (hexadecyl alcohol, cetyl alcohol, palmityl alcohol) ethoxylated with 107 EO,
hexadecan-1-ol (hexadecyl alcohol, cetyl alcohol, palmityl alcohol) ethoxylated with 108 EO,
hexadecan-1-ol (hexadecyl alcohol, cetyl alcohol, palmityl alcohol) ethoxylated with 109 EO,
hexadecan-1-ol (hexadecyl alcohol, cetyl alcohol, palmityl alcohol) ethoxylated with 110 EO,
octadecan-1-ol (octadecyl alcohol, stearyl alcohol) ethoxylated with 50 EO,
octadecan-1-ol (octadecyl alcohol, stearyl alcohol) ethoxylated with 51 EO,
octadecan-1-ol (octadecyl alcohol, stearyl alcohol) ethoxylated with 52 EO,
octadecan-1-ol (octadecyl alcohol, stearyl alcohol) ethoxylated with 53 EO,
octadecan-1-ol (octadecyl alcohol, stearyl alcohol) ethoxylated with 54 EO,
octadecan-1-ol (octadecyl alcohol, stearyl alcohol) ethoxylated with 55 EO,
octadecan-1-ol (octadecyl alcohol, stearyl alcohol) ethoxylated with 56 EO,
octadecan-1-ol (octadecyl alcohol, stearyl alcohol) ethoxylated with 57 EO,
octadecan-1-ol (octadecyl alcohol, stearyl alcohol) ethoxylated with 58 EO,
octadecan-1-ol (octadecyl alcohol, stearyl alcohol) ethoxylated with 59 EO,
octadecan-1-ol (octadecyl alcohol, stearyl alcohol) ethoxylated with 60 EO,
octadecan-1-ol (octadecyl alcohol, stearyl alcohol) ethoxylated with 90 EO,
octadecan-1-ol (octadecyl alcohol, stearyl alcohol) ethoxylated with 91 EO,
octadecan-1-ol (octadecyl alcohol, stearyl alcohol) ethoxylated with 92 EO,
octadecan-1-ol (octadecyl alcohol, stearyl alcohol) ethoxylated with 93 EO,
octadecan-1-ol (octadecyl alcohol, stearyl alcohol) ethoxylated with 94 EO,
octadecan-1-ol (octadecyl alcohol, stearyl alcohol) ethoxylated with 95 EO,
octadecan-1-ol (octadecyl alcohol, stearyl alcohol) ethoxylated with 96 EO,
octadecan-1-ol (octadecyl alcohol, stearyl alcohol) ethoxylated with 97 EO,
octadecan-1-ol (octadecyl alcohol, stearyl alcohol) ethoxylated with 98 EO,
octadecan-1-ol (octadecyl alcohol, stearyl alcohol) ethoxylated with 99 EO,
octadecan-1-ol (octadecyl alcohol, stearyl alcohol) ethoxylated with 100 EO,
octadecan-1-ol (octadecyl alcohol, stearyl alcohol) ethoxylated with 101 EO,
octadecan-1-ol (octadecyl alcohol, stearyl alcohol) ethoxylated with 102 EO,
octadecan-1-ol (octadecyl alcohol, stearyl alcohol) ethoxylated with 103 EO,
octadecan-1-ol (octadecyl alcohol, stearyl alcohol) ethoxylated with 104 EO,
octadecan-1-ol (octadecyl alcohol, stearyl alcohol) ethoxylated with 105 EO,
octadecan-1-ol (octadecyl alcohol, stearyl alcohol) ethoxylated with 106 EO,
octadecan-1-ol (octadecyl alcohol, stearyl alcohol) ethoxylated with 107 EO,
octadecan-1-ol (octadecyl alcohol, stearyl alcohol) ethoxylated with 108 EO,
octadecan-1-ol (octadecyl alcohol, stearyl alcohol) ethoxylated with 109 EO,
octadecan-1-ol (octadecyl alcohol, stearyl alcohol) ethoxylated with 110 EO is used.

6. The composition according to claim 1, wherein the at least one polyol is selected from 1,2-propylene glycol, glycerol, butylene glycols such a 1,2-butylene glycol, 1,3-butylene glycol 1,4-butylene glycol, pentylene glycols hexanediols, hexanetriols, 1,2-octanediol, 1,8-octanediol, dipropylene glycol, tripropylene glycol, diglycerol, triglycerol, erythritol, sorbitol, PEG-3, PEG-4, PEG-6, PEG-7, PEG-8, or mixtures of the aforementioned substances.

7. The composition according to claim 1, wherein the at least one oil is selected from natural hydrocarbons; synthetic hydrocarbons; the benzoic acid esters of linear or branched C₈₋₂₂ alkanols; fatty alcohols with 6-30 carbon atoms, which are unsaturated or branched and saturated or branched and unsaturated; triglycerides of linear or branched, saturated or unsaturated optionally hydroxylated C₈₋₃₀ fatty acids; the dicarboxylic acid esters of linear or branched C₂-C₁₀ alkanols; the esters of the linear or branched, saturated or unsaturated fatty alcohols with 2-30 carbon atoms with linear or branched, saturated or unsaturated fatty acids with 2-30 carbon atoms, which may be hydroxylated; the addition products of 1 to 5 propylene oxide units onto monovalent or polyvalent C₈-₂₂ alkanols; the addition products of at least 6 ethylene oxide and/or propylene oxide units onto monovalent or polyvalent C₃-₂₂ alkanols; the C₈-C₂₂ fatty alcohol esters of monovalent or polyvalent C₂-C₇ hydroxycarboxylic acids; the symmetrical, asymmetrical or cyclic esters of carbonic acid with C₃-₂₂ alkanols, C₃-₂₂ alkanediols or C₃-₂₂ alkanetriols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols; silicone oils; or mixtures of the aforementioned substances.

8. The composition according to claim 1, comprising at least one lamellar phase.

9. A kit for oxidative color change of keratinic fibers comprising two separate compositions (A) and (B), wherein
the composition (B) is an oxidative composition comprising:
from about 50-96% by weight water,
from about 0.5-20% by weight hydrogen peroxide,
at least one linear saturated 1-alkanol with 12-30 carbon atoms,
at least one mono- or diester of ethylene glycol with palmitic acid and/or stearic acid as well as mixtures thereof,
at least one ethoxylated fatty alcohol of the formula (III) 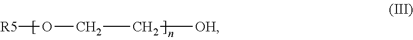
where R5 stands for a saturated or unsaturated, branched or unbranched C₈-C₂₄ alkyl group and n stands for an integer from 10 to 30,
at least one ethoxylated fatty alcohol of formula (IV) 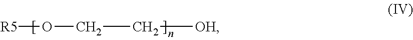
where R5 stands for a saturated or unsaturated, branched or unbranched C₈-C₂₄ alkyl group and n stands for an integer from about 50 to about 150,
at least one polyol selected from C₂-C₉ alkanols with 2-6 hydroxyl groups, polyethylene glycols with 3-20 ethylene oxide units, or mixtures thereof, and
at least one oil,
wherein all the quantitative amounts are based on the weight of the oxidative composition, and
the composition (A) is present in the form of a soap-based gel and comprises:
from about 25-85% by weight water,
ammonia,
from about 5-20% by weight of at least one salt of a C₁₂-C₂₂ fatty acid
from about 2-20% by weight of at least one polyethylene glycol ether of a linear saturated or unsaturated C₁₀-C₁₈ alkanol with 1-5 ethylene oxide units in the molecule,
from about 0-1% by weight of at least one oil,
optionally at least one organic solvent, selected from monovalent to tetravalent C₂-C₆ alcohols
optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule and
optionally at least one oxidative dye precursor
wherein composition (A) has a pH in the range of from about 8 to about 11.5 measured at 20° C.,
wherein all the quantitative amounts are based on the weight of the composition (A).

10. A method for oxidative color change of keratinic fibers, comprising the following method steps:
providing an oxidative composition (B) comprising
from about 50 to about 96% by weight water,
from about 0.5 to about 20% by weight hydrogen peroxide,
at least one linear saturated 1-alkanol with 12 to 30 carbon atoms,
at least one mono- or diester of ethylene glycol with palmitic acid and/or stearic acid as well as mixtures thereof,
at least one ethoxylated fatty alcohol of the formula (III) 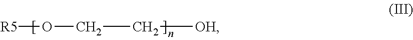
where R5 stands for a branched or unbranched, saturated or unsaturated C₈-C₂₄ alkyl group and n stands for an integer from 10 to 30,
at least one ethoxylated fatty alcohol of formula (IV) 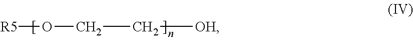
where R5 stands for a branched or unbranched, saturated or unsaturated C₈-C₂₄ alkyl group and n stands for an integer from 50 to 150,
at least one polyol selected from C₂-C₉ alkanols with 2-6 hydroxyl groups, polyethylene glycols with 3-20 ethylene oxide units, or mixtures thereof, and
at least one oil,
wherein all the quantitative amounts are based on the weight of the oxidative composition, and
providing a composition (A), which is in the form of a soap-based gel and comprises
from about 25-85% by weight water,
ammonia,
from about 5-20% by weight of at least one salt of a C₁₂-C₂₂ fatty acid,
from about 2-20% by weight of at least one polyethylene glycol ether of a linear saturated or unsaturated C₁₀-C₁₈ alkanol with 1 to 5 ethylene oxide units in the molecule,
a total of from about 0-1% by weight of at least one oil,
at least one organic solvent, selected from monovalent to tetravalent C₂-C₆ alcohols,
optionally from about 0.1-3% by weight of an anionic surfactant selected from alkyl sulfates, alkyl ether sulfates and ether carboxylic acids with 10 to 20 carbon atoms in the alkyl group and up to 16 glycol ether groups in the molecule and
optionally at least one oxidative dye precursor
wherein composition (A) has a pH in the range of from about 8 to about 11.5 measured at 20° C.,
wherein all the quantitative amounts are based on the weight of the composition (A);
preparing a mixture of the aforementioned composition (B) and the aforementioned composition (A),
immediately thereafter distributing the ready-to-use agent on the fibers,
leaving the agent on the fibers for a period of from about 1 to about 60 minutes, then immediately thereafter rinsing the remaining agent out of the fibers and optionally drying the fibers.

11. The oxidation composition of claim 1, comprising from about 80-90% by weight water, based on the weight of the oxidative composition.

12. The oxidation composition of claim 1, wherein the at least one linear saturated 1-alkanol with 12-30 carbon atoms is present in a total amount of from about 2-8% by weight, based on the weight of the oxidative composition.

13. The oxidation composition of claim 1, wherein the esters of ethylene glycol with palmitic acid and/or stearic acid are present in a total amount of from about 0.01-1% by weight, based on the weight of the oxidative composition.

14. The oxidation composition of claim 1, wherein in the at least one ethoxylated fatty alcohol of the formula (III) R5 stands for a saturated unbranched C₁₆ or C₁₈ alkyl group and n stands for an integer from 15 to 20

15. The oxidation composition of claim 1, wherein the at least one ethoxylated fatty alcohol of formula (III) is present in a total amount of from about 0.25-2% by weight, based on the weight of the oxidative composition.

16. The oxidation composition of claim 1, wherein in the at least one ethoxylated fatty alcohol of formula (IV) R5 stands for a saturated unbranched C₁₆ or C₁₈ alkyl group and n stands for an integer from about 80 to about 120.

17. The oxidation composition of claim 1, wherein the at least one ethoxylated fatty alcohol of formula (IV) is present in a total amount of from about 0.03-0.3% by weight, based on the weight of the oxidative composition.

18. The oxidation composition of claim 1, wherein the at least one polyol selected from C₂-C₉ alkanols with 2-6 hydroxyl groups, polyethylene glycols with 3-20 ethylene oxide units, or mixtures thereof is present in a total amount of from about 1-10% by weight, based on the weight of the oxidative composition.

19. The oxidation composition of claim 1, wherein the at least one oil is present in a total amount of from about 0.1-5% by weight, based on the weight of the oxidative composition.

20. The oxidation composition of claim 1, comprising:
from about 80-90% by weight water,
the at least one linear saturated 1-alkanol with 12-30 carbon atoms present in an amount of from about 3 to 6.5% by weight,
the at least one mono- or diester of ethylene glycol with palmitic acid and/or stearic acid as well as mixtures thereof present in an amount of from about 0.1 to 0.8% by weight,
the at least one ethoxylated fatty alcohol of the formula (III) where R5 stands for a saturated unbranched C₁₆ or C₁₈ alkyl group and n stands for an integer from 15 to 20, wherein the at least one ethoxylated fatty alcohol of formula (III) is present in a total amount of from about 0.5 to 1.5% by weight,
the at least one ethoxylated fatty alcohol of formula (IV) where R5 stands for a saturated unbranched C₁₆ or C₁₈ alkyl group and n stands for an integer from 80 to about 120, wherein the at least one ethoxylated fatty alcohol of formula (IV) is present in a total amount of from about 0.1-0.2% by weight,
the at least one polyol is present in an amount of from about 3-6% by weight, and
the at least one oil is present in an amount of from about 0.5-2.5% by weight,
wherein all the quantitative amounts are based on the weight of the oxidative composition.
